(19) United States
(12) Patent Application Publication
(10) Pub. No.: US 2022/0183578 A1
(43) Pub. Date: Jun. 16, 2022

(54) VASCULAR ENDOTHELIAL FUNCTION EVALUATION DEVICE, METHOD, AND VASCULAR ENDOTHELIAL FUNCTION EVALUATION SYSTEM

(71) Applicant: HUMAN LINK CO., LTD., Asaka-shi, Saitama (JP)

(72) Inventor: Katsutoshi TAGAMI, Asaka-shi, Saitama (JP)

(21) Appl. No.: 17/432,660

(22) PCT Filed: Feb. 27, 2019
(86) PCT No.: PCT/JP2019/007499

§ 371 Date: Aug. 20, 2021

(51) Int. Cl. A61B 5/021; A61B 5/0235; A61B 5/00

(52) U.S. Cl. CPC A61B 5/02116; A61B 5/742; A61B 5/0235; A61B 5/02141

(57) ABSTRACT

Provided is a technology of allowing evaluation of a vascular endothelial function stably and accurately at relatively low introduction cost, regardless of an examiner's skill. Using a pressure applying belt including an inflatable bladder, which is similar to a cuff for blood pressure measurement, the vascular endothelial function is evaluated. As pre-processing, while an arm tightening force by the pressure applying belt is changed, a maximum pulse wave pressure being a an air pressure inside the inflatable bladder at the time when a pulse wave amplitude is maximum is identified, and the pulse wave amplitude at this time is recorded. Next, occlusion processing of causing occlusion in the arm and releasing the occlusion is performed. Then, as post-processing, the pulse wave amplitude is measured while the air pressure inside the inflatable bladder is kept at the maximum pulse wave pressure, and the pulse wave amplitude at the time of its peak is recorded.

### TECHNICAL FIELD

The present invention relates to a technology for evaluating a vascular endothelial function.

### BACKGROUND ART

Blood vessels have significant influence on human health. For example, when the blood vessel has high elasticity, blood pressure is lowered.

The elasticity of the blood vessel is not invariant. For example, the elasticity of the blood vessel increases through production of nitric oxide (NO) in the blood vessel. Nitric oxide has a function of relaxing vascular smooth muscle forming the blood vessel, and hence an increase of the nitric oxide in blood causes such improvement of the elasticity of the blood vessel. The blood vessel has a function of self-producing nitric oxide. Endothelial cells are present in the vascular endothelium on an inner surface of the blood vessel, and the endothelial cells produce the nitric oxide. When blood flows through the blood vessel, the vascular endothelium is applied with a stress like being rubbed by the blood. When this stress called “shear stress” is applied to the vascular endothelium, the endothelial cells stimulated with the shear stress produce the nitric oxide. When the nitric oxide produced as described above increases in the blood, the elasticity of the blood vessel consequently increases.

The elasticity of the blood vessel is a useful index for evaluating the degree of health of the blood vessel, but how much ability the blood vessel has to produce the nitric oxide, and further how much degree the elasticity of the blood vessel increases when the shear stress is caused are also useful indices for evaluating the degree of health of the blood vessel. Evaluating how much degree the elasticity of the blood vessel increases when the shear stress is caused means, in other words, evaluating a vascular endothelial function.

As a technology for evaluating the vascular endothelial function, in more detail, evaluating how much degree the elasticity of the blood vessel increases when the shear stress is caused, there is known a method called “flow-mediated dilation (FMD)” test. An apparatus for performing the FMD is already available for sale, and is also in practical use.

Briefly speaking, the most widespread apparatus for performing the FMD includes an ultrasonic diagnostic device and a tourniquet. The ultrasonic diagnostic device includes a display for allowing a diameter of an arterial vessel of a subject to be checked substantially in real time through visual observation of a moving image. The tourniquet is for use to occlude arterial blood in the artery of the subject (in general, the tourniquet is equivalent to a pneumatic cuff to be used in blood pressure measurement).

When this apparatus is used to evaluate the vascular endothelial function, first, the arm artery of the subject in a resting state is measured through visual observation based on a blood vessel image displayed on the display of the ultrasonic diagnostic device. Next, the artery is occluded for 5 minutes by the tourniquet at a pressure at which the subject's arm artery is brought into a completely occluded state (this pressure is, in general, a pressure higher than the subject's systolic blood pressure by 50 mmHg). After an elapse of 5 minutes under this state, the occlusion of the artery by the tourniquet is released, and the diameter of the subject's arm artery is measured again with the ultrasonic diagnostic device. In the artery which has been brought into the occluded state for 5 minutes and then subjected to release of the occlusion, a flow of blood larger than that in the case of the resting state is caused. As a result, a shear stress larger than that in the case of the resting state is caused. The artery which has obtained more elasticity than that in the case of the resting state due to an action of a larger amount of nitric oxide as compared to that in the case of the resting state, which is caused by the shear stress larger than that in the case of the resting state, is increased in diameter as compared to that in the case of the resting state. After the release of the occlusion, the diameter of the artery at the time of its peak is measured as a diameter after the release of the occlusion. Some period of time is required from when the shear stress is applied to the vascular endothelium to when the endothelial cells release nitric oxide into the blood, and also from when the concentration of the nitric oxide in the blood rises to when the elasticity of the blood vessel increases. Further, the half-life of nitric oxide in blood is from 3 seconds to 6 seconds. In view of such circumstances, although there are individual differences, the timing at which the artery has the maximum diameter after the release of the occlusion of the artery is a predetermined time point within a time range of from an elapse of 45 seconds to an elapse of 120 seconds, in general, from an elapse of 45 seconds to an elapse of 60 seconds from the release of the occlusion. An examiner observes the artery displayed as a moving image on the display of the ultrasonic diagnostic device, and measures the maximum value of the diameter of the blood vessel, which appears at some time point within a period of from an elapse of 45 seconds to an elapse of 120 seconds from the release of the occlusion of the artery.

Then, after the diameters of the blood vessel before and after the occlusion are measured, the vascular endothelial function is evaluated based on a relationship between those two values. For example, when the diameter of the blood vessel before the occlusion is represented by D_{B}, and the diameter of the blood vessel after the occlusion is represented by D_{A}, D_{A}/D_{B} or (D_{A}−D_{B})/D_{B} is calculated so that the vascular endothelial function is evaluated. No matter which calculation is performed, as the value is larger (in short, as D_{A} being the diameter of the blood vessel after the occlusion is larger), it means that a larger amount of nitric oxide is produced in the blood and thus the elasticity of the blood vessel is increased, and hence it is evaluated that the vascular endothelial function is better.

When the FMD is performed, as described above, occlusion and release of the occlusion are performed. Those operations are performed so as to grasp a difference between the characteristic of the blood vessel at the time when blood flows through the artery in an amount at a normal time and the elasticity of the blood vessel at the time when blood flows through the artery in an amount larger than that at the normal time. Further, in the general FMD, a physical quantity related to the blood vessel, which is to be used to grasp the difference, is the diameter of the blood vessel.

Meanwhile, as the physical quantity, other than the diameter of the blood vessel, a pulse wave (pulse wave amplitude) is sometimes used. When the blood vessel has elasticity, a certain volume of blood pushed out by the heartbeat to the blood vessel flows through the blood vessel while expanding the blood vessel, and hence the blood velocity is decreased. However, when the blood vessel has no elasticity, the certain volume of blood smoothly flows through the blood vessel without expanding the blood vessel, and hence the blood velocity is relatively increased. That is, the pulse wave is a physical quantity related to the elasticity of the blood vessel, and hence, even through use of the pulse wave, the vascular endothelial function can be indirectly evaluated. The FMD utilizing the pulse wave as described above is generally called “ezFMD”, and is proposed in a paper of “The Japanese Journal of Medical Instrumentation, 2012, Vol. 82, No. 3.”

According to this paper, the ezFMD is carried out as follows.

First, a pneumatic cuff is wrapped around a subject's arm. The following processing is carried out under this state. Under this state, the systolic blood pressure and the diastolic blood pressure of the subject in the resting state, and the pulse wave are measured. The pulse wave amplitude at this time corresponds to a pulse wave amplitude before the occlusion. Then, similarly to a case of carrying out an oscillometric method to be used when blood pressure is measured, an air pressure inside of the cuff is raised to reach a value higher than the subject's systolic blood pressure, and then is gradually reduced. Then, in a process of gradually reducing the pressure, the pulse wave is measured. The pulse wave amplitude is maximum when the air pressure inside of the cuff is close to an intermediate point between the systolic blood pressure and the diastolic blood pressure. This is because, when the air pressure inside of the cuff has this extent of magnitude, there is achieved a state in which the internal pressure and the external pressure of the blood vessel are substantially balanced, in other words, no tension is applied to the vascular wall. Then, the pulse wave amplitude at the time of its peak in the pressure reducing process is identified as a pulse wave amplitude after the occlusion.

When the method of the ezFMD is carried out, the vascular endothelial function is evaluated based on a relationship between the above-mentioned two pulse wave amplitudes. For example, when the pulse wave amplitude in the resting state before the occlusion is represented by P_{B}, and the pulse wave amplitude at the time point of its peak after the occlusion is represented by P_{A}, P_{A}/P_{B} or (P_{A}−P_{B}) P_{B} is calculated so that the vascular endothelial function is evaluated. No matter which calculation is performed, as the value is larger (in short, as P_{A} being the pulse wave amplitude after the occlusion is larger), it means that a larger amount of nitric oxide is produced in the blood and thus the elasticity of the blood vessel is increased, and hence it is evaluated that the vascular endothelial function is better.

### SUMMARY OF INVENTION

### Technical Problem

However, each of the general FMD and the ezFMD described above has problems.

First, in the general FMD, it can be pointed out that an ultrasonic diagnostic device which often costs several tens of millions of yen is required, and hence the introduction cost is high. Further, in the FMD, the examiner measures the diameter of the artery while viewing the display of the ultrasonic diagnostic device, and hence the evaluation result of the vascular endothelial function varies depending on the examiner's skill. In addition, it is considered that, for example, as in a case in which a larger force is required to further stretch a stretched rubber as compared to a force required to stretch an unstretched rubber, there occurs a phenomenon in which, in a case in which the internal pressure of the blood vessel is large, that is, in a case in which the subject has high blood pressure and a large tension is applied to the vascular wall even under the resting state, the diameter of the blood vessel is not increased so much even when the internal pressure further acts on the artery by the blood. That is, it is considered that the degree of elasticity of the blood vessel (or how easy the diameter of the blood vessel changes) varies depending on the level of the blood pressure of the subject, and hence there is a fear in that the evaluation result of the vascular endothelial function obtained by the FMD lacks accuracy particularly when the subject has high blood pressure.

On the other hand, the ezFMD does not use the ultrasonic diagnostic device, and further a device for measuring the pulse wave is normally quite less expensive than the ultrasonic diagnostic device. Accordingly, the introduction cost is lower than that of the general FMD. Further, in the ezFMD, the vascular endothelial function is evaluated through use of the pulse wave amplitude which has no room for entry of the examiner's skill, and hence the evaluation result obtained by the ezFMD is likely to become stable. In addition, in the ezFMD, in particular, measurement of the pulse wave amplitude after the release of the occlusion is performed under a state in which no tension is applied to the blood vessel, and hence a result of the measurement is always stable whether the subject has high blood pressure or low blood pressure.

However, even the ezFMD has points for improvement. As described in the description of the general FMD, although there are individual differences, the diameter of the artery after the release of the occlusion of the artery is maximum at some time point from an elapse of 45 seconds to an elapse of 120 seconds from the release of the occlusion. This timing is the timing at which the blood vessel has the highest elasticity. In the general FMD, in order not to overlook this timing, the diameter of the blood vessel is continuously observed for the entire period of time from an elapse of 45 seconds to an elapse of 120 seconds from the release of the occlusion, in which there is a possibility that the blood vessel has the highest elasticity. In contrast, in the ezFMD, the pulse wave amplitude is measured at a time point at which the air pressure inside of the cuff substantially matches a subject's mean blood pressure. In the ezFMD, it is assumed that the pulse wave amplitude is maximum at this time, but if this fact is true, the reason therefor is highly likely to be because a state in which no tension is applied to the vascular wall is achieved at this time point. That is, although the blood vessel achieves the highest elasticity at some timing from an elapse of 45 seconds to an elapse of 120 seconds from the release of the occlusion, in the ezFMD configured to measure the pulse wave after the release of the occlusion at a time point at which the air pressure inside of the cuff substantially matches the subject's mean blood pressure, there is no guarantee that the blood vessel has the highest elasticity due to the action of the nitric oxide at this time point at which the pulse wave after the release of the occlusion is measured. The air pressure inside of the cuff is reduced irrespective of the amount of the nitric oxide in the blood or the degree of improvement of the elasticity of the blood vessel based on the nitric oxide in the blood, and hence, unless extremely lucky, the time point at which the air pressure inside of the cuff matches the subject's mean blood pressure is normally earlier or later than the timing at which the elasticity of the blood vessel of the subject is most increased. Accordingly, regarding the measurement result of the pulse wave after the release of the occlusion in the ezFMD, assuming that, in the blood vessel of the same subject, the same blood vessel elasticity is achieved after an elapse of the same time period when the same shear stress is caused in the blood vessel, the evaluation result of the vascular endothelial function has stability in a case in which the measurement is performed several times for the same subject. However, in a case in which the vascular endothelial function is evaluated for a large number of subjects, there is a high possibility that the accuracy of the evaluation result of the vascular endothelial function cannot be guaranteed.

The present invention has been made to solve the above-mentioned problems, and has an object to provide a technology for allowing a vascular endothelial function to be evaluated stably and accurately, regardless of the examiner's skill.

### Solution to Problem

In order to solve the above-mentioned problems, the inventor of the subject application proposes the following invention.

According to the invention of the subject application, there is provided a vascular endothelial function evaluation device, which is configured to form a vascular endothelial function evaluation system in combination with: a tightener including: a belt having a length which allows the belt to be wrapped around a predetermined part of any one of limbs; fixing means for fixing the belt under a state in which the belt is wrapped around the predetermined part of the limb; and an inflatable bladder which is provided on the belt, and is configured to apply a predetermined tightening pressure to the predetermined part of the limb by tightening the predetermined part of the limb through loading of a gas inside of the inflatable bladder under a state in which the belt wrapped around the predetermined part of the limb is fixed by the fixing means; a pressure varying device configured to set a pressure of the gas inside of the inflatable bladder to a desired pressure; and a pulse wave measuring device configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter.

The vascular endothelial function evaluation device (hereinafter sometimes simply referred to as “evaluation device”) includes control means for controlling the pressure varying device, the control means being configured to receive the pulse wave data from the pulse wave measuring device. Further, the control means is configured to execute: pre-processing of controlling the pressure varying device so as to cause the pressure varying device to execute a first phase being processing of changing the pressure of the gas inside of the inflatable bladder to pass a range in which the pulse wave amplitude is assumed to be maximum, receiving the pulse wave data a plurality of times from the pulse wave measuring device while the pressure inside of the inflatable bladder is changed by the first phase to identify a maximum pulse wave pressure being the pressure of the gas inside of the inflatable bladder at a time when the pulse wave amplitude is maximum, and recording the pulse wave amplitude at the time when the maximum pulse wave pressure is caused; occlusion processing of controlling the pressure varying device so as to cause the pressure varying device to execute a second phase being processing of maintaining the pressure of the gas inside of the inflatable bladder for at least 3 minutes or more at a pressure equal to or higher than a pressure at which occlusion occurs in the artery of the limb to which the tightener is fixed, and then decreasing the pressure of the gas inside of the inflatable bladder; and post-processing of controlling the pressure varying device so as to cause the pressure varying device to execute a third phase of maintaining the pressure of the gas inside of the inflatable bladder within a range of 15 mmHg around the maximum pulse wave pressure until at least 90 seconds elapse from when the processing of decreasing the pressure of the gas inside of the inflatable bladder is ended in the occlusion processing, receiving the pulse wave data a plurality of times from the pulse wave measuring device while the pressure inside of the inflatable bladder is maintained in a state of the third phase, and recording a maximum pulse wave amplitude based on pieces of pulse wave data received the plurality of times.

The evaluation device is configured to form the vascular endothelial function evaluation system in combination with the tightener, the pressure varying device, and the pulse wave measuring device. As a device for measuring a physical quantity related to the elasticity of the blood vessel, instead of using the ultrasonic diagnostic device, the pulse wave measuring device configured to measure the pulse wave is used. Accordingly, similarly to the case of the ezFMD, the introduction cost is relatively low. Further, similarly to the case of the ezFMD, this evaluation device evaluates the vascular endothelial function through use of the pulse wave amplitude which has no room for entry of the examiner's skill, and hence the evaluation result obtained by the evaluation device is likely to become stable.

The tightener to be used in combination with the evaluation device of the invention of the subject application includes: a belt having a length which allows the belt to be wrapped around a predetermined part of any one of limbs; fixing means for fixing the belt under a state in which the belt is wrapped around the predetermined part of the limb; and an inflatable bladder which is provided on the belt, and is configured to apply a predetermined tightening pressure to the predetermined part of the limb by tightening the predetermined part of the limb through loading of a gas inside of the inflatable bladder under a state in which the belt wrapped around the predetermined part of the limb is fixed by the fixing means. For example, this tightener can be configured similarly to a cuff to be generally used for measuring blood pressure, or can be configured similarly to a pneumatic belt to be used in a strength training method called Kaatsu training (trademark). The number of tighteners may be only one, and the limb to which the tightener is attached is generally an arm. The width and the length of the tightener are selected as appropriate depending on the limb to which the tightener is attached.

The pressure varying device to be used in combination with the evaluation device of the invention of the subject application is configured to set the pressure of the gas inside of the inflatable bladder included in the tightener to a desired pressure. Specifically, the pressure varying device can be formed of a pump, a valve, or the like. The gas to be injected into the inflatable bladder is generally air, although not limited thereto.

The pulse wave measuring device to be used in combination with the evaluation device of the invention of the subject application is configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter. The pulse wave amplitude is measured in the vicinity of the part at which the tightener is fixed or on the distal end side of the limb from the part. This is for allowing the pulse wave measuring device to detect the pulse wave of the artery to which the tightening effect by the tightener is exerted. In the pulse wave measuring device as well as the above-mentioned pressure varying device, a part of the configurations of those devices may be included in the vascular endothelial function evaluation device as a part of the vascular endothelial function evaluation device.

The evaluation device in the invention of the subject application includes control means for controlling the pressure varying device, the control means being configured to receive the pulse wave data from the pulse wave measuring device.

In this case, the control means executes the following pre-processing, occlusion processing, and post-processing in the stated order.

In the pre-processing, main control means controls the pressure varying device so as to cause the pressure varying device to execute a first phase being processing of changing the pressure of the gas inside of the inflatable bladder to pass a range in which the pulse wave amplitude is assumed to be maximum, receives the pulse wave data a plurality of times from the pulse wave measuring device while the pressure inside of the inflatable bladder is changed by the first phase to identify a maximum pulse wave pressure being the pressure of the gas inside of the inflatable bladder at a time when the pulse wave amplitude is maximum, and records the pulse wave amplitude at the time when the maximum pulse wave pressure is caused. This processing corresponds to the measurement of the pulse wave amplitude in the resting state in the ezFMD, and is executed in the resting state. However, in the pre-processing of the invention of the subject application, the measurement of the pulse wave amplitude is performed a plurality of times (for example, multiple times from about several times to about several tens of times in 1 second) while the pressure of the gas inside of the inflatable bladder is changed to pass a range in which the pulse wave amplitude is assumed to be maximum. Then, the main control means records the pulse wave amplitude at its peak in the changed pulse wave amplitude as the pulse wave amplitude equivalent to the pulse wave amplitude in the resting state in the ezFMD. Similarly to the case of the ezFMD, at this time, it is considered that the pressure of the gas inside of the inflatable bladder or the tightening force applied to the limb by the tightener is possibly about the subject's mean blood pressure, and there is achieved a state in which no tension is applied to the vascular wall at this time. In the evaluation device of the invention of the subject application, the blood pressure may be monitored, but the monitoring of the blood pressure is not always required. In the pre-processing in the invention of the subject application, it is only required that the pulse wave amplitude at the time when the amplitude is maximum be recorded, and that the maximum pulse wave pressure being the gas pressure inside of the inflatable bladder at this time be identified. It is referred to above that “the pressure of the gas inside of the inflatable bladder or the tightening force applied to the limb by the tightener” because, when a cuff of a sphygmomanometer is wrapped around an arm, in general, no tightening force is applied to the limb by the cuff under a state in which no air is supplied to the cuff, but in the case of the tightener to be used in combination with the evaluation device of the invention of the subject application, in some cases, the tightener applies some extent of tightening force to the predetermined part of the limb under a state in which the tightener is attached to the limb (for example, a tightening force of 30 mmHg or less: this tightening force is referred to as “wearing pressure”), and in such a case, a pressure obtained by adding the wearing pressure to the air pressure inside of the inflatable bladder corresponds to the tightening force to be actually applied to the limb by the tightener.

The occlusion processing to be executed next is a processing of controlling the pressure varying device so as to cause the pressure varying device to execute a second phase being processing of maintaining the pressure of the gas inside of the inflatable bladder for at least 3 minutes or more at a pressure equal to or higher than a pressure at which occlusion occurs in the artery of the limb to which the tightener is fixed, and then decreasing the pressure of the gas inside of the inflatable bladder. This occlusion processing corresponds to occlusion and release of the occlusion in the ezFMD. The occlusion time period is set to 3 minutes or more because this time period is sufficient for causing a change of the elasticity of the blood vessel before and after the occlusion. This time period may be larger, for example, 5 minutes similarly to the case of the general FMD or the ezFMD. In this manner, the evaluation result obtained by the evaluation device of the invention of the subject application can be more easily compared with the evaluation result obtained by the general FMD or the ezFMD. The occlusion is caused in the limb when the pressure of the gas inside of the inflatable bladder or the tightening force applied to the limb by the tightener exceeds the subject's systolic blood pressure. When the occlusion is caused in the general FMD or the ezFMD, the air pressure inside of the cuff is brought to a pressure exceeding the systolic blood pressure by 50 mmHg because a margin of 50 mmHg is given in order to reliably cause the occlusion in consideration of the above. The pressure of the gas inside of the inflatable bladder at the time when the occlusion is caused in the limb in the invention of the subject application may be set to a pressure exceeding the systolic blood pressure by 50 mmHg similarly thereto, and further, whether or not there is a wearing pressure, a sum of the wearing pressure and the pressure of the gas inside of the inflatable bladder may be set to a pressure exceeding the systolic blood pressure by 50 mmHg. In this manner, the evaluation result obtained by the evaluation device of the invention of the subject application can be easily compared with the evaluation result obtained by the general FMD or the ezFMD. However, the pressure of the gas inside of the inflatable bladder at the time when the second phase is executed is only required to be a pressure that causes occlusion in the artery, and whether or not the pressure is constant does not matter.

In the post-processing to be executed next, under a state in which the pressure inside of the inflatable bladder is kept within a range of 15 mmHg around the maximum pulse wave pressure, the measurement of the pulse wave amplitude is performed a plurality of times (for example, multiple times from about several times to about several tens of times in 1 second). Then, the main control means records the pulse wave amplitude at its peak in the changed pulse wave amplitude as the pulse wave amplitude equivalent to the pulse wave amplitude after the release of the occlusion in the ezFMD. When the pulse wave amplitude is measured in the post-processing in the subject application, in a case in which the wearing pressure is the same in the pre-processing and in the post-processing (this condition is normally satisfied as long as the change of the wearing pressure due to the re-wrapping of the tightener to the limb is not performed between the pre-processing and the post-processing), and the air pressure inside of the inflatable bladder is the same as the maximum pulse wave pressure determined in the pre-processing, the tightening force applied to the arm by the tightener is kept within a range having only a difference of 7.5 mmHg at maximum from the tightening force applied to the limb by the tightener at a time point at which the pulse wave amplitude at its peak is measured in the pre-processing, regardless of the magnitude of the wearing pressure (also including a case in which the wearing pressure is 0 mmHg). That is, in the third phase, the tightening force applied to the limb by the tightener is always close to the tightening force applied to the limb by the tightener at the time point at which the pulse wave amplitude at its peak is measured in the pre-processing, and hence there is achieved a state in which no tension is always applied or at least only a small tension is applied to the vascular wall of the artery. Further, the third phase is continued until at least 90 seconds elapse from the end of the processing of decreasing the pressure of the gas inside of the inflatable bladder, that is, the processing of releasing the occlusion in the occlusion processing. The time period from when the occlusion is released to when the artery attains the highest elasticity is, as described above, from an elapse of 45 seconds to an elapse of 120 seconds from the release of the occlusion, and there are individual differences. However, most subjects attain the highest artery elasticity until 90 seconds elapse from the release of the occlusion. Accordingly, as long as the third phase is started within 45 seconds from when the occlusion is ended in the occlusion processing, in theory, the pulse wave amplitude at the moment at which the blood vessel has the highest elasticity can be grasped by measuring the pulse wave amplitude at which the amplitude is maximum. When more perfection is aimed at, in the third phase, keeping the pressure inside of the inflatable bladder within a range of 15 mmHg around the maximum pulse wave pressure may be started before 45 seconds elapse from the end of the occlusion in the occlusion processing, and the pressure inside of the inflatable bladder may be maintained until at least 120 seconds elapse. However, when the tightening to the limb in the post-processing is performed immediately after the end of the occlusion in the occlusion processing, there is a fear in that the increase of the shear stress caused by the release of the occlusion in the blood vessel of the artery becomes insufficient. Accordingly, it is preferred that the tightening in the post-processing be started after about 15 seconds or more elapse from when the occlusion is ended in the occlusion processing.

As is apparent from the description above, in the pre-processing executed in the invention of the subject application, how much pressure should be applied to the limb in order to prevent tension from being applied to the vascular wall of the artery is identified by identifying the maximum pulse wave pressure. Then, the pulse wave amplitude at its peak, which is recorded in the pre-processing, has a highly-stable objective numerical value which is not affected by the level of the blood pressure of the subject under a state in which no tension is applied to the vascular wall of the artery. Further, in the post-processing executed in the invention of the subject application, which is executed after occlusion and release of the occlusion similar to those executed in the general FMD or the ezFMD, while a state in which no tension is applied or a small tension is applied to the vascular wall of the artery is kept, the pulse wave amplitude is continuously observed in the period of time in which the artery is assumed to have the highest elasticity, and hence the pulse wave amplitude at the moment at which the artery has the highest elasticity can be grasped without fail. Then, the pulse wave amplitude grasped at this time is the pulse wave amplitude in the state in which no tension or a small tension is applied to the vascular wall of the artery, and hence has a highly-stable objective numerical value which is not affected or substantially not affected by the level of the blood pressure of the subject.

Accordingly, the evaluation of the vascular endothelial function to be performed through use of the pulse wave amplitude grasped in the pre-processing and the post-processing in the subject application is more accurate and stable than the evaluation of the vascular endothelial function to be performed in the general FMD or the ezFMD. The same holds true even when the pulse wave amplitude is similarly measured a plurality of times for the same subject, or when the pulse wave amplitude is similarly measured a plurality of times for a plurality of subjects.

That is, when the effects of the vascular endothelial function evaluation device according to the invention of the subject application are summarized, the vascular endothelial function can be evaluated stably and accurately with a relatively low introduction cost, regardless of the examiner's skill.

In the vascular endothelial function evaluation device of the invention of the subject application, the pulse wave amplitude is recorded when the pre-processing and the post-processing are each executed. The subject's vascular endothelial function is evaluated based on those two pieces of pulse wave amplitude, but this evaluation may be performed by, for example, a doctor or other examiner. Meanwhile, the control means may be configured to generate result data of a numerical value of a result of performing predetermined calculation based on the pulse wave amplitude recorded in the pre-processing and the pulse wave amplitude recorded in the post-processing. The result data may be generated immediately after the post-processing is ended, or may be generated at a timing not immediately after the post-processing is ended, for example, when the input for prompting the generation of the result data is performed with respect to the evaluation device. As a matter of course, the evaluation device may be configured to output the result data. Whether the examiner performs the evaluation or the evaluation device performs the evaluation, when the pulse wave amplitude recorded in the pre-processing is represented by P_{B}, and the pulse wave amplitude recorded in the post-processing is represented by P_{A}, P_{A}/P_{B} or (P_{A}−P_{B}) P_{B} can be calculated so that the evaluation result of the vascular endothelial function is obtained.

As described above, in the third phase executed at the time of the post-processing, the pressure of the gas inside of the inflatable bladder is kept at a pressure within a range of 15 mmHg around the maximum pulse wave pressure. In the third phase, the pressure of the gas inside of the inflatable bladder may fluctuate within the range of 15 mmHg around the maximum pulse wave pressure. Meanwhile, in the third phase, the pressure of the gas inside of the inflatable bladder may be kept constant within the range of 15 mmHg around the maximum pulse wave pressure. When the latter case is adopted, although based on the premise that the subject does not move his or her limb to which the tightener is attached during the execution of the post-processing, the pressure varying device is not required to be driven during the execution of the post-processing. Accordingly, the processing to be performed by the evaluation device in the invention of the subject application can be simplified.

In the third phase, the pressure of the gas inside of the inflatable bladder may be kept constant at the maximum pulse wave pressure. In this case, while the third phase is executed, in theory, a state in which no tension is applied to the vascular wall of the artery is maintained. Accordingly, in theory, the influence of blood pressure with respect to the pulse wave amplitude to be measured at its peak in the post-processing can be completely eliminated.

As described above, the pulse wave measuring device is configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter. The pulse wave measuring device may be adapted to measure the pressure of the gas inside of the inflatable bladder as the predetermined parameter. This means that so-called cuff pulse wave measurement is adopted in the invention of the subject application. In this case, the place at which the pulse wave is measured is a part to which the tightener is attached of the limb to which the tightener is attached.

Meanwhile, a device for measuring a pulse wave at a fingertip or other portion is publicly known or well known, and is commercially available. The pulse wave can be measured with such a device, and when such a pulse wave measuring method is adopted in the invention of the subject application, the portion at which the pulse wave is measured is not limited to the part to which the tightener is attached of the limb to which the tightener is attached.

The inventor of the subject application further proposes the following vascular endothelial function evaluation system as one mode of the invention of the subject application. The effect of this vascular endothelial function evaluation system is the same as the effect of the evaluation device according to the invention of the subject application.

As an example, the vascular endothelial function evaluation system includes: a tightener including: a belt having a length which allows the belt to be wrapped around a predetermined part of any one of limbs; fixing means for fixing the belt under a state in which the belt is wrapped around the predetermined part of the limb; and an inflatable bladder which is provided on the belt, and is configured to apply a predetermined tightening pressure to the predetermined part of the limb by tightening the predetermined part of the limb through loading of a gas inside of the inflatable bladder under a state in which the belt wrapped around the predetermined part of the limb is fixed by the fixing means; a pressure varying device configured to set a pressure of the gas inside of the inflatable bladder to a desired pressure; and a pulse wave measuring device configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter.

Further, the vascular endothelial function evaluation system includes control means for controlling the pressure varying device, the control means being configured to receive the pulse wave data from the pulse wave measuring device. The control means is configured to execute: pre-processing of controlling the pressure varying device so as to cause the pressure varying device to execute a first phase being processing of changing the pressure of the gas inside of the inflatable bladder to pass a range in which the pulse wave amplitude is assumed to be maximum, receiving the pulse wave data a plurality of times from the pulse wave measuring device while the pressure inside of the inflatable bladder is changed by the first phase to identify a maximum pulse wave pressure being the pressure of the gas inside of the inflatable bladder at a time when the pulse wave amplitude is maximum, and recording the pulse wave amplitude at the time when the maximum pulse wave pressure is caused; occlusion processing of controlling the pressure varying device so as to cause the pressure varying device to execute a second phase being processing of maintaining the pressure of the gas inside of the inflatable bladder for at least 3 minutes or more at a pressure equal to or higher than a pressure at which occlusion occurs in the artery of the limb to which the tightener is fixed, and then decreasing the pressure of the gas inside of the inflatable bladder; and post-processing of controlling the pressure varying device so as to cause the pressure varying device to execute a third phase of maintaining the pressure of the gas inside of the inflatable bladder within a range of 15 mmHg around the maximum pulse wave pressure until at least 90 seconds elapse from when the processing of decreasing the pressure of the gas inside of the inflatable bladder is ended in the occlusion processing, receiving the pulse wave data a plurality of times from the pulse wave measuring device while the pressure inside of the inflatable bladder is maintained in a state of the third phase, and recording a maximum pulse wave amplitude based on pieces of pulse wave data received the plurality of times.

The inventor of the subject application further proposes the following vascular endothelial function evaluation method as one mode of the invention of the subject application. The effect of this vascular endothelial function evaluation method is the same as the effect of the evaluation device according to the invention of the subject application.

As an example, there is provided a vascular endothelial function evaluation method of a vascular endothelial function evaluation device, the vascular endothelial function evaluation device being configured to form a vascular endothelial function evaluation system in combination with: a tightener including: a belt having a length which allows the belt to be wrapped around a predetermined part of any one of limbs; fixing means for fixing the belt under a state in which the belt is wrapped around the predetermined part of the limb; and an inflatable bladder which is provided on the belt, and is configured to apply a predetermined tightening pressure to the predetermined part of the limb by tightening the predetermined part of the limb through loading of a gas inside of the inflatable bladder under a state in which the belt wrapped around the predetermined part of the limb is fixed by the fixing means; a pressure varying device configured to set a pressure of the gas inside of the inflatable bladder to a desired pressure; and a pulse wave measuring device configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter, the vascular endothelial function evaluation device including control means for controlling the pressure varying device, the control means being configured to receive the pulse wave data from the pulse wave measuring device. The control means is configured to execute the vascular endothelial function evaluation method.

The vascular endothelial function evaluation method executed by the control means includes: pre-processing of controlling the pressure varying device so as to cause the pressure varying device to execute a first phase being processing of changing the pressure of the gas inside of the inflatable bladder to pass a range in which the pulse wave amplitude is assumed to be maximum, receiving the pulse wave data a plurality of times from the pulse wave measuring device while the pressure inside of the inflatable bladder is changed by the first phase to identify a maximum pulse wave pressure being the pressure of the gas inside of the inflatable bladder at a time when the pulse wave amplitude is maximum, and recording the pulse wave amplitude at the time when the maximum pulse wave pressure is caused; occlusion processing of controlling the pressure varying device so as to cause the pressure varying device to execute a second phase being processing of maintaining the pressure of the gas inside of the inflatable bladder for at least 3 minutes or more at a pressure equal to or higher than a pressure at which occlusion occurs in the artery of the limb to which the tightener is fixed, and then decreasing the pressure of the gas inside of the inflatable bladder; and post-processing of controlling the pressure varying device so as to cause the pressure varying device to execute a third phase of maintaining the pressure of the gas inside of the inflatable bladder within a range of 15 mmHg around the maximum pulse wave pressure until at least 90 seconds elapse from when the processing of decreasing the pressure of the gas inside of the inflatable bladder is ended in the occlusion processing, receiving the pulse wave data a plurality of times from the pulse wave measuring device while the pressure inside of the inflatable bladder is maintained in a state of the third phase, and recording a maximum pulse wave amplitude based on pieces of pulse wave data received the plurality of times.

### BRIEF DESCRIPTION OF DRAWINGS

FIG. 1 is a view for illustrating an overall configuration of a vascular endothelial function evaluation system according to a first embodiment of the present invention;

FIG. 2 is a perspective view for illustrating a pressure applying belt included in the vascular endothelial function evaluation system illustrated in FIG. 1;

FIG. 3 is a hardware configuration diagram of an evaluation device included in the vascular endothelial function evaluation system illustrated in FIG. 1;

FIG. 4 is a diagram for illustrating a hardware configuration of a control unit of the evaluation device included in the vascular endothelial function evaluation system illustrated in FIG. 1;

FIG. 5 is a block diagram for illustrating functional blocks formed inside of the control unit;

FIG. 6 is a time chart for illustrating a pressure of air inside of an inflatable bladder at the time when second preliminary processing and pre-processing are executed in the evaluation system illustrated in FIG. 1;

FIG. 7 is a graph for showing a relationship between a pulse wave amplitude and a pressure of air inside of the inflatable bladder at the time when the pre-processing is executed in the evaluation system illustrated in FIG. 1;

FIG. 8 is a time chart for illustrating a pressure of air inside of the inflatable bladder at the time when occlusion processing is executed in the evaluation system illustrated in FIG. 1; and

FIG. 9 are time charts for illustrating a pressure of air inside of the inflatable bladder at the time when post-processing is executed in the evaluation system illustrated in FIG. 1.

### DESCRIPTION OF EMBODIMENTS

Now, one embodiment of the present invention is described with reference to the drawings.

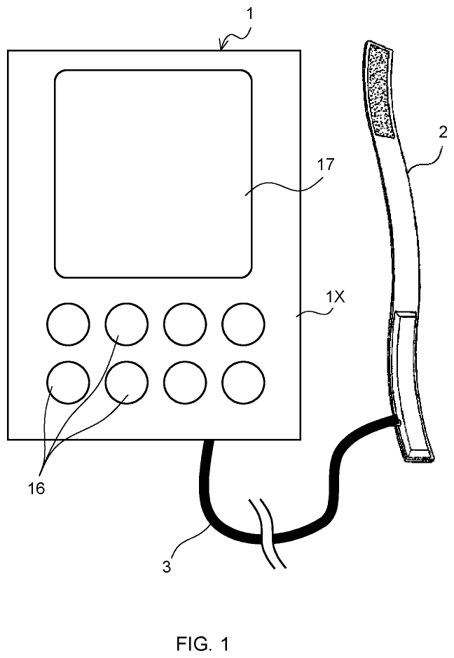

FIG. 1 shows an overall vascular endothelial function evaluation system according to the one embodiment of the present invention.

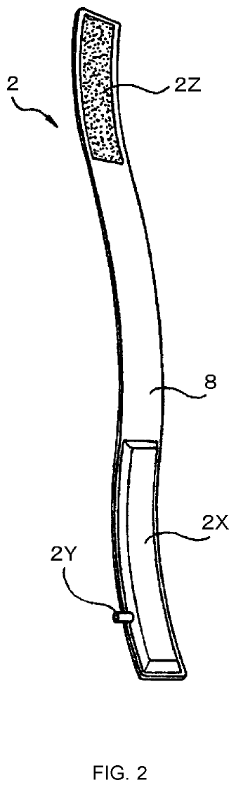

This vascular endothelial function evaluation system includes a vascular endothelial function evaluation device 1 (hereinafter sometimes simply referred to as “evaluation device 1”), and a pressure applying belt 2 whose details are illustrated in FIG. 2. The pressure applying belt 2 corresponds to a tightener referred to in the subject application.

First, a configuration of the pressure applying belt 2 is described.

The pressure applying belt 2 is controlled by the evaluation device 1 so that such a tightening force that causes restriction of blood, which for example is occlusion, is applied to a subject's limb to which the pressure applying belt 2 is attached. In this embodiment, the limb to which the pressure applying belt 2 is attached is a subject's arm, but the limb may be a leg.

The pressure applying belt 2 may be one. The present invention is not limited thereto, but, in this embodiment, one pressure applying belt 2 is included in the vascular endothelial function evaluation system.

The pressure applying belt 2 can be configured similarly to a cuff included in a sphygmomanometer, or can be configured similarly to a pneumatic belt to be used in Kaatsu training (trademark).

The configuration of the pressure applying belt 2 as an example is described.

The pressure applying belt 2 includes an elongated tightening band 8 formed into a belt shape. The tightening band 8 is made of a material that does not practically stretch in a length direction of the tightening band 8, for example, a woven fabric woven with appropriate threads. The pressure applying belt 2 has a length set to the extent that there is still some margin after the pressure applying belt 2 is wrapped once around a predetermined part of the arm to which the pressure applying belt 2 is expected to be attached, for example, a predetermined part in the vicinity of the base of the arm.

The width of the tightening band 8 is set to an appropriate width such that the tightening band 8 is thin to the extent that the tightening band 8 is prevented from overlapping the muscle belly when being attached to the predetermined part in the vicinity of the base of the arm, and is thick to the extent that the tightening band 8 is prevented from biting into the arm to cause pain to the subject. However, the width of the tightening band 8 may be larger, and it is not always required to prevent the tightening band 8 from overlapping the muscle belly when the tightening band 8 is attached to the arm.

For example, on an inner surface of the tightening band 8, an airtight inflatable bladder 2X is mounted. The inflatable bladder 2X is made of a material that can withstand an air pressure of about 400 mmHg. The inflatable bladder 2 may be made of a stretchable material such as natural rubber, or may be made of a resin or other material that is substantially non-stretchable. The inflatable bladder 2X is connected in a communicating state to a coupling tube 2Y being, for example, a resin tube. The inflatable bladder 2X is connected via the coupling tube 2Y to a distal end of a rubber tube 3 which is made of, for example, rubber and is a tube whose proximal end is to be connected to the evaluation device 1. The inflatable bladder 2X corresponds to an inflatable bladder in the invention of the subject application. The inflatable bladder 2X in this embodiment is provided on the inner surface of the tightening band 8, but the inflatable bladder 2X may be arranged inside of a bag-shaped tightening band 8.

On an inner side of the tightening band 8, a fixing portion 2Z is further provided so as to fix a diameter of a loop formed by the tightening band 8 when the tightening band 8 of the pressure applying belt 2 is wrapped at a predetermined position of the subject's arm. As long as this fixing is allowed, the fixing portion 2Z may have any configuration. The fixing portion 2Z in this embodiment is a hook-and-loop fastener, although not limited thereto. When the tightening band 8 is wrapped around the subject's arm from the lower side of FIG. 2 so that the fixing portion 2Z is fixed to an outer surface of the tightening band 8, the diameter of the loop formed by the tightening band 8 is fixed.

Under a state in which the pressure applying belt 2 is fixed to an appropriate part of the subject's arm, air is supplied into or discharged from the inflatable bladder 2X of the pressure applying belt 2 via the rubber tube 3 by the evaluation device 1. The pressure of the air causes the pressure applying belt 2 to apply a tightening force at an appropriate pressure with respect to the subject's arm to which the pressure applying belt 2 is attached.

Next, the evaluation device 1 is described.

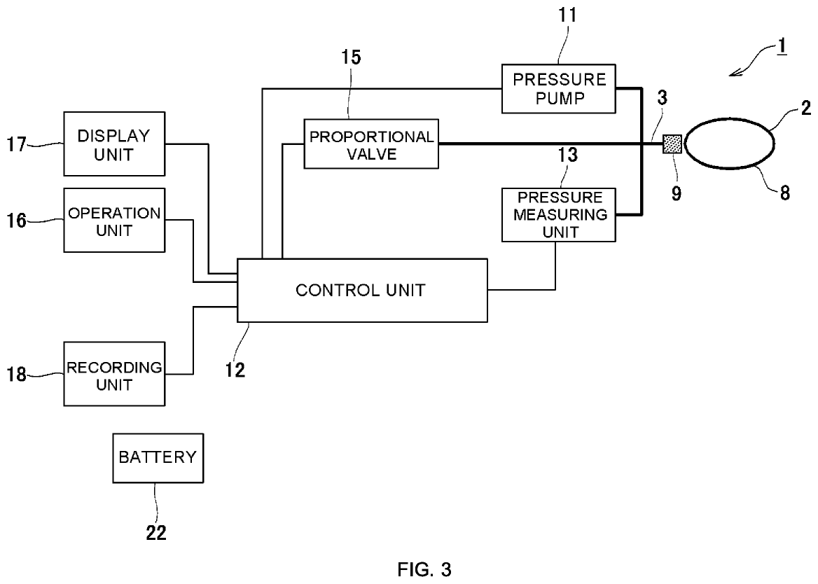

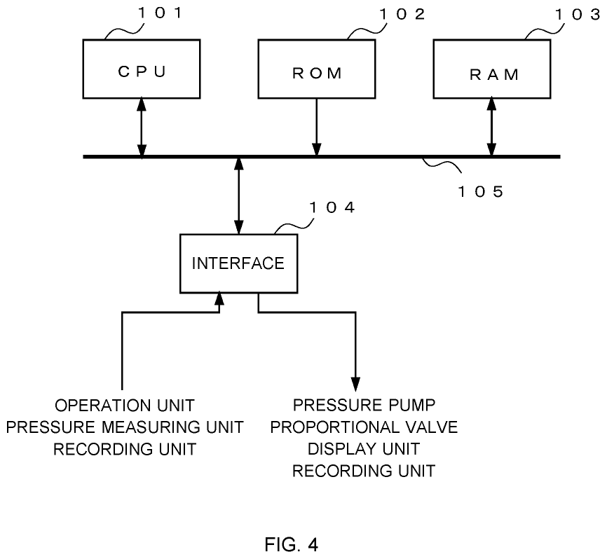

The specific configuration of the evaluation device 1 is illustrated in FIG. 1, FIG. 3, and FIG. 4.

The evaluation device 1 has a function of supplying air into the inflatable bladder 2X of the pressure applying belt 2 wrapped around the subject's arm, thereby causing the pressure applying belt 2 to tighten the subject's arm. In this manner, the evaluation device 1 can cause, for example, occlusion of the arm artery by applying an appropriate tightening force to the subject's arm, and can also release the occlusion.

Further, as described later, the evaluation device 1 has a function of detecting a pulse wave amplitude of the subject's arm to which the pressure applying belt 2 is attached. Further, the evaluation device 1 has a function of evaluating a subject's vascular endothelial function based on the pulse wave amplitude measured as described later.

The evaluation device 1 of this embodiment is formed by, as illustrated in FIG. 1, mounting or incorporating various components to a hollow casing 1X being, for example, a resin box, although not always limited thereto.

The casing 1X of the evaluation device 1 has an operation unit 16 provided in any form, for example, in a form of buttons or a dial. The operation unit 16 is configured to generate data based on an operation performed thereon. The operation unit 16 is connected to a control unit 12 so that required data can be input to the control unit 12. When the operation unit 16 is operated, as described later, a series of evaluation processing steps including pre-processing, occlusion processing, and post-processing can be started or ended. At least input of data required therefor can be carried out through the operation unit 16.

A display unit 17 is provided to the casing 1X. The display unit 17 is configured to display characters or images, and includes a display, for example, a liquid crystal display (LCD). The display unit 17 displays, for example, the content input through the operation of the operation unit 16, and, in this embodiment, the content of the evaluation of the subject's vascular endothelial function, although not limited thereto. The display unit 17 performs appropriate display in addition to the above based on data generated by the control unit 12 to be described later. The display unit 17 may have a touch panel function. In this manner, at least a part of the functions of the operation unit 16 can be implemented by the display unit 17. It is also possible to omit the operation unit 16 so that all of the functions of the operation unit 16 may be implemented by the display unit 17 having the touch panel function.

As described above, as illustrated in FIG. 3, the pressure applying belt 2 to be worn on at least one of the subject's arms can be connected to the evaluation device 1.

The pressure applying belt 2 is connected to the evaluation device 1 as required via the rubber tube 3 being a tube serving as a connecting member. The rubber tube 3 is required as many as the number of pressure applying belts, and only one rubber tube 3 is provided in this embodiment because one pressure applying belt 2 is used. One end of each rubber tube 3 is connected to the inflatable bladder 2X of the pressure applying belt 2 via the coupling tube 2Y, and another end of each rubber tube 3 is connected to the evaluation device 1. A valved coupler 9 is mounted to a distal end portion of each rubber tube 3, and the inflatable bladder 2X of each pressure applying belt 2 is connected to this valved coupler 9.

As illustrated in FIG. 3, the evaluation device 1 includes a pressure pump 11. The pressure pump 11 is an air pump. The pressure pump 11 is connected to the rubber tube 3 connected to the pressure applying belt 2 as described above, and can supply air into the inflatable bladder 2X of the pressure applying belt 2 via the rubber tube 3 and also discharge air inside of the inflatable bladder 2X. In this embodiment, a gas to be injected into and discharged from the inflatable bladder 2X is air, although not limited thereto.

The pressure pump 11 in this embodiment is incorporated in the casing 1X. However, the pressure pump 11 may be present outside of the casing 1X or outside of the evaluation device 1 as a unit separate from the evaluation device 1.

The evaluation device 1 further includes a pressure measuring unit 13. The pressure measuring unit 13 is formed of a sensor capable of measuring a pressure of a gas. The pressure measuring unit 13 is configured to measure the pressure of the air inside of the rubber tube 3 to indirectly measure the pressure of the inflatable bladder 2X, to thereby further indirectly measure a pressure applied to the subject's arm at this time point by the pressure applying belt 2. The pressure measuring unit 13 in this embodiment is connected to a branch tube branched from the rubber tube 3, although not always limited thereto. The pressure measuring unit 13 measures the pressure of the air inside of the branch tube so as to indirectly measure the pressure of the gas (in this embodiment, air) inside of the inflatable bladder 2X. The pressure measuring unit 13 generates pressure data being data of the measured air pressure.

The pressure measuring unit 13 is further connected to the control unit 12. The pressure data generated by the pressure measuring unit 13 is transmitted to the control unit 12. The control unit 12 uses the pressure data to control the pressure pump 11 as described later. As described later, the control unit 12 further detects a subject's pulse wave (pulse wave amplitude) from the pressure data. The pressure data is successively generated, and is transmitted to the control unit substantially in real time. The pressure data can be generated, for example, from several times to several tens of times in 1 second. The number of times to generate the pressure data can be determined depending on, for example, a length of a time period required to smoothly discharge air in the pre-processing to be described later (time period in which a first phase referred to in the subject application is executed). When this time period is short, it is required to increase the number of times to generate the pressure data per second. As the number of times to generate the pressure data per second is increased, it is possible to more reliably measure the pulse wave amplitude based on the variation of the pressure of the air inside of the inflatable bladder 2X, in more detail, measure the pulse wave amplitude at the time of its peak. However, in general, when the pressure data is generated about several tens of times in 1 second, this object can be achieved, and hence it is generally not required to generate a larger number of pressure data than that. It is also possible to change the number of times to generate the pressure data per second in each of the pre-processing, the occlusion processing, and the post-processing to be described later, but in this embodiment, the number of times to generate the pressure data per second is the same in all of the above-mentioned three processing steps, although not limited thereto.

The evaluation device 1 further includes a proportional valve 15. The proportional valve 15 is a control valve capable of performing proportional adjustment of the pressure of the air inside of the rubber tube 3. With the presence of the proportional valve 15, even when, for example, the subject moves to cause variation in thickness of the muscle of the arm to which the pressure applying belt 2 is attached, a pressure force applied to the pressure applying belt 2 is kept constant through proportional-integral-differential (PID) control. The proportional valve 15 in this embodiment is connected to a branch tube branched from the proximal end side of the rubber tube 3, which is different from the branch tube connected to the pressure measuring unit 13, so as to adjust the pressure of the air inside of this branch tube, although not always limited thereto. The proportional valve 15 is connected to the control unit 12, and the operation of the proportional valve 15 is controlled based on data from the control unit 12. However, the proportional valve 15 may be regarded as a part of the pressure pump 11. For example, the proportional valve 15 may play a role of discharging air from the inflatable bladder 2X. In this case, the pressure of the air inside of the inflatable bladder 2X is controlled through cooperation by the pressure pump 11 for increasing the pressure and the proportional valve 15 for decreasing the pressure.

The control unit 12 included in the evaluation device is a computer, and is configured to control the entire evaluation device 1. For example, the control unit 12 controls the drive of the pressure pump 11 (and the proportional valve 15). Further, as described later, the control unit 12 detects the pulse wave amplitude, and records the detected pulse wave amplitude. Further, as described later, the control unit 12 evaluates the subject's vascular endothelial function.

The control unit 12 of the evaluation device 1 includes hardware illustrated in FIG. 4. The hardware included in the control unit 12 includes a CPU 101 being an arithmetic logic unit, a ROM 102 storing a program for determining processing to be executed by the CPU 101 and data required for executing the program, a RAM 103 for providing a working space when the CPU 101 executes the program, and an interface 104 for connecting an external device and the CPU 101 or the like to each other. Further, the CPU 101, the ROM 102, the RAM 103, and the interface 104 are connected to each other via a bus 105. The program and the data stored in the ROM 102 at least include a computer program or data required for generating functional blocks to be described later inside of the control unit 12. This computer may be configured to generate the functional blocks to be described later alone, or may be configured to generate the functional blocks in cooperation with an OS or other program. Various types of data are stored in the RAM 103, but the function of the RAM 103 may be implemented by a recording unit 18 to be described later, or the function of the recording unit 18 may be implemented by the RAM 103. Further, the control unit 12 may include a hard disk drive (HDD) or other large-capacity recording medium, and the large-capacity recording medium may implement at least a part of the functions of the ROM 102 and the RAM 103. The interface 104 is connected to the pressure measuring unit 13, the pressure pump 11, the proportional valve 15, the operation unit 16, the display unit 17, and the recording unit (described later).

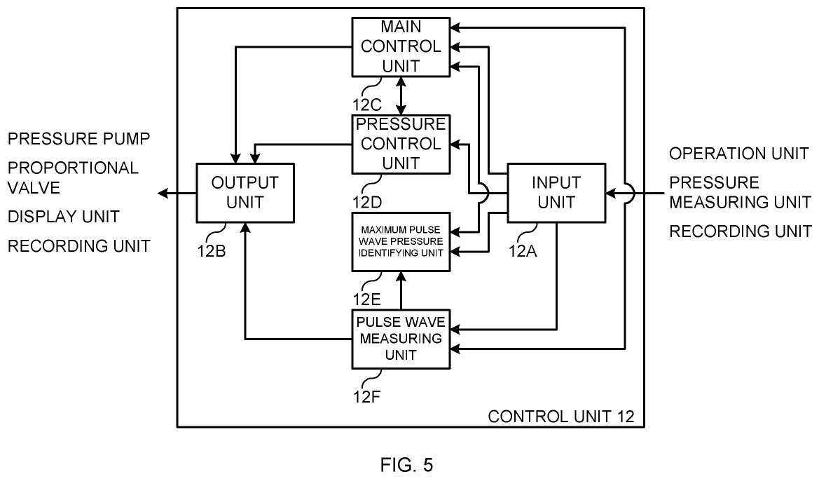

Through execution of the above-mentioned program, the functional blocks as illustrated in FIG. 5 are generated in the control unit 12.

The functional blocks to be generated are an input unit 12A, an output unit 12B, a main control unit 12C, a pressure control unit 12D, a maximum pulse wave pressure identifying unit 12E, and a pulse wave measuring unit 12F.

The input unit 12A is configured to receive external data input with respect to the control unit 12. The input unit 12A transmits the received data to an appropriate functional block. For example, the input unit 12A receives data input from the operation unit 16, and transmits the data to the main control unit 12C. The input unit 12A further receives the pressure data from the pressure measuring unit 13, and transmits the pressure data to the pressure control unit 12D, the maximum pulse wave pressure identifying unit 12E, and the pulse wave measuring unit 12F.

In some cases, the recording unit 18, which is to be described later, transmits pulse wave amplitude data, which is also to be described later, to the input unit 12A. The input unit 12A which has received the pulse wave amplitude data transmits the pulse wave amplitude data to the main control unit 12C.

The main control unit 12C is configured to control the entire evaluation device 1. The main control unit 12C performs this control based on the data input from the operation unit 16. This control to be performed by the main control unit 12C includes, for example, switching on and off the power of the evaluation device 1, processing of starting and ending a series of evaluation processing steps including the pre-processing, the occlusion processing, and the post-processing to be described later, and processing of starting each of the pre-processing, the occlusion processing, and the post-processing. When the main control unit 12C starts the evaluation processing, the main control unit 12C transmits data indicating the start to the pressure control unit 12D and the pulse wave measuring unit 12F.

Further, when the main control unit 12C executes each of the pre-processing, the occlusion processing, and the post-processing, the main control unit 12C transmits, to the pressure control unit 12D, pressure control data being data of a function of time and pressure (or data of a time chart), which indicates how the air pressure inside of the inflatable bladder 2X is required to be maintained when each of the pre-processing, the occlusion processing, and the post-processing is performed. In this embodiment, the pressure control data is transmitted also before first preliminary processing and second preliminary processing to be described later are executed.

As described later, the main control unit 12C receives, from the maximum pulse wave pressure identifying unit 12E, maximum pulse wave pressure data being data identifying the maximum pulse wave pressure, which is also to be described later. As described later, the maximum pulse wave pressure data is used when the pressure control data is generated while the post-processing is executed.

Further, as described later, when the pre-processing and the post-processing are executed or after those processing steps are executed, in some cases, the main control unit 12C receives, from the pulse wave measuring unit 12F, pulse wave amplitude data being data identifying the pulse wave amplitude. The main control unit 12C which has received the pulse wave amplitude data transmits the pulse wave amplitude data to the output unit 12B.

In some cases, the main control unit 12C further receives, from the input unit 12A, two pieces of pulse wave amplitude data being paired. The main control unit 12C which has received those pieces of pulse wave amplitude data has a function of generating evaluation data indicating the evaluation result of the subject's vascular endothelial function based on the pulse wave amplitude data. After generating the evaluation data, the main control unit 12C transmits the evaluation data to the output unit 12B.

The pressure control unit 12D is configured to control the pressure pump 11 to control the pressure of the air inside of the inflatable bladder 2X provided in the pressure applying belt 2, to thereby control the pressure to be applied to the subject's arm by the pressure applying belt 2. The above-mentioned functions are exerted when any of the pre-processing, the occlusion processing, and the post-processing is executed. The control of the pressure of the air inside of the inflatable bladder 2X of the pressure applying belt 2 in each of the pre-processing, the occlusion processing, and the post-processing is executed in accordance with the pressure control data received by the pressure control unit 12D from the main control unit 12C before each of those three processing steps is executed.

As described above, the pressure control unit 12D receives the pressure data from the input unit 12A, and monitors, substantially in real time, based on the received pressure data, the pressure of the air inside of the inflatable bladder 2X provided in the pressure applying belt 2 at this time point. The pressure control unit 12D includes a timer (not shown), and generates first control data being data for driving the pressure pump 11 so as to maintain the pressure of the air inside of the inflatable bladder 2X at the time point identified by the timer at a pressure identified by the above-mentioned pressure control data. When this first control data is received, the pressure pump 11 is driven in accordance with the first control data, and the pressure of the air inside of the inflatable bladder 2X is changed or maintained as appropriate.

Further, the pressure control unit 12D generates second control data for controlling the proportional valve 15 to be described later. The proportional valve 15 which has received the second control data as described later is driven as described later in accordance with an instruction given by the second control data. The pressure control unit 12D transmits the generated first control data and second control data to the output unit 12B. How to generate the first control data and the second control data is described in detail later. When there is no proportional valve 15, as a matter of course, the pressure control unit 12D is not required to have the function of generating the second control data.

The pulse wave measuring unit 12F receives the pressure data from the input unit 12A. The pressure data is transmitted to the pulse wave measuring unit 12F when the pre-processing and the post-processing are executed. The pulse wave measuring unit 12F detects the pulse wave amplitude after performing a publicly-known or well-known processing, for example, removing noise from the pressure data, and further detects, from the detected pulse wave amplitude, the timing at which the pulse wave amplitude is maximum and the pulse wave amplitude at this time point. The pulse wave amplitude data being data identifying the pulse wave amplitude at the time of the maximum amplitude while the pre-processing and the post-processing are executed is transmitted from the pulse wave measuring unit 12F to the main control unit 12C, for example, at a time point at which the pre-processing is ended and a time point at which the post-processing is ended. Further, timing data being data indicating the timing at which the pulse wave amplitude is maximum in the pre-processing is transmitted from the pulse wave measuring unit 12F to the maximum pulse wave pressure identifying unit 12E. In this embodiment, the pulse wave measuring unit 12F detects, even when the second preliminary processing to be described later is executed, the timing at which the pulse wave amplitude is maximum and the pulse wave amplitude at this time point, and transmits the pulse wave amplitude data identifying the pulse wave amplitude at the time of its peak to the main control unit 12C.

The maximum pulse wave pressure identifying unit 12E is configured to identify the maximum pulse wave pressure. The maximum pulse wave pressure identifying unit 12E functions, that is, identifies the maximum pulse wave pressure when the pre-processing is executed. As described above, the maximum pulse wave pressure identifying unit 12E receives the pressure data from the input unit 12A. The maximum pulse wave pressure identifying unit 12E monitors, similarly to the pressure control unit 12D, the pressure of the air inside of the inflatable bladder 2X based on the pressure data. Meanwhile, as described above, the maximum pulse wave pressure identifying unit 12E receives the timing data from the pulse wave measuring unit 12F. The maximum pulse wave pressure identifying unit 12E identifies the pressure of the air inside of the inflatable bladder 2X at the timing identified by the timing data as the maximum pulse wave pressure being the pressure of the air inside of the inflatable bladder 2X at the time when the pulse wave amplitude is maximum. After identifying the maximum pulse wave pressure, the maximum pulse wave pressure identifying unit 12E generates the maximum pulse wave pressure data indicating the maximum pulse wave pressure, and transmits the maximum pulse wave pressure data to the main control unit 12C.

The output unit 12B is configured to output data from the control unit 12 to the outside. The output unit 12B transmits the received data to an appropriate device outside of the control unit 12.

As described above, in some cases, the output unit 12B receives the evaluation data indicating the evaluation result of the subject's vascular endothelial function from the main control unit 12C. When receiving this evaluation data, the output unit 12B has a function of generating image data of an image including, for example, characters, for use to display the evaluation data on the display unit 17. The generated image data is transmitted from the output unit 12B to the display unit 17. The display unit 17 which has received the image data displays the image based on the image data.

In some cases, the output unit 12B further receives the first control data and the second control data from the pressure control unit 12D. When receiving the first control data, the output unit 12B transmits the first control data to the pressure pump 11, and when receiving the second control data, the output unit 12B transmits the second control data to the proportional valve 15. The pressure pump 11 which has received the first control data is driven in accordance with the first control data, and the proportional valve 15 which has received the second control data is driven in accordance with the second control data.

As described above, in some cases, the output unit 12B further receives the pulse wave amplitude data from the main control unit 12C. When receiving the pulse wave amplitude data, the output unit 12B transmits the pulse wave amplitude data to the recording unit 18. The recording unit 18 is, for example, a part of the RAM 103, and has a function of recording data. At least the pulse wave amplitude data is recorded in the recording unit 18. The recording unit 18 in this embodiment records two pieces of pulse wave amplitude data generated at the time of pre-processing and post-processing which are performed in series for the same subject, in a state of being linked with each other or as a pair, although not limited thereto. In the recording unit 18, other data, for example, data identifying to which subject the pieces of pulse wave amplitude data being paired belong may be recorded. Further, in the recording unit 18, appropriate data such as a history of operations performed on the operation unit 16 and a history of occurrence of abnormality may be recorded. In some cases, the pieces of pulse wave amplitude data being paired and recorded in the recording unit 18 are read out as described later, and transmitted to the input unit 12A via the interface 104.

Further, a battery 22 is mounted on the evaluation device 1. The battery 22 is for use to drive the control unit 12, the pressure measuring unit 13, the proportional valve 15, the operation unit 16, the display unit 17, the recording unit 18, and the like forming the evaluation device 1. The battery may be a publicly-known or well-known battery, and the function, the usage, and the like of the battery 22 in this embodiment are also publicly known or well known. Thus, detailed description thereof is omitted.

Next, a usage method and an operation of the vascular endothelial function evaluation system are described.

First, a doctor or other examiner operates a power switch included in the operation unit 16 of the evaluation device 1 to turn on the power and activate the evaluation device 1. Data input through the operation unit 16 is transmitted to the main control unit 12C via the interface 104 and the input unit 12A. The main control unit 12C which has received this data turns on the power of the evaluation device 1.

Around the time when the power of the evaluation device 1 is turned on, the examiner fixes the pressure applying belt 2 at a predetermined position in the vicinity of a proximal end of one of the subject's arms. When the pressure applying belt 2 is fixed, the inflatable bladder 2X is brought into abutment against the arm. The pressure applying belt 2 is wrapped around the arm from the lower side of FIG. 2, and the fixing portion 2Z is fixed to the outer surface of the tightening band 8 so that the pressure applying belt 2 is removably fixed to the subject's arm. However, under this state, the pressure applying belt 2 is loosely attached to the arm. Under this state, the pressure applying belt 2 is connected to the evaluation device 1 via the tube 3. In this manner, the pressure of the air inside of the inflatable bladder 2X of the pressure applying belt 2 can be controlled by the evaluation device 1.

After that, in order to evaluate the subject's vascular endothelial function, the pre-processing, the occlusion processing, and the post-processing are sequentially executed in the stated order, but prior thereto, two processing steps are executed. The two processing steps are referred to as “first preliminary processing” and “second preliminary processing.” However, the first preliminary processing and the second preliminary processing are not always required.

Briefly speaking, the first preliminary processing is, processing of adjusting a wearing pressure to be applied to the subject's arm by the pressure applying belt 2, which is to be described later, to an appropriate magnitude.

When the first preliminary processing is to be executed, the examiner operates the operation unit 16 to input data indicating the execution of the first preliminary processing. This data is transmitted from the operation unit 16 to the main control unit 12C via the interface 104 and the input unit 12A. The main control unit 12C which has received the data transmits, to the pressure control unit 12D, the data indicating the execution of the first preliminary processing and the pressure control data being the data of a function of time and pressure, which indicates how the air pressure inside of the inflatable bladder 2X is required to be maintained when the first preliminary processing is performed.

The pressure control unit 12D generates the first control data so that the pressure of the air inside of the inflatable bladder 2X of the pressure applying belt 2 follows the instruction given by the received pressure control data. The generated first control data is transmitted to the pressure pump 11 via the output unit 12B, and thus the pressure pump 11 is driven. Under the control of the control unit 12, the pressure pump 11 supplies air to the inflatable bladder 2X of the pressure applying belt 2, and first sets the pressure of the air inside of the inflatable bladder 2X to a relatively low pressure of, in general, from about 10 mmHg to about 15 mmHg, for example, about 13 mmHg. At this time, a small tightening force applied to the arm by the pressure applying belt 2 is useful to prevent the pressure applying belt 2 from rotating around the arm.

In this embodiment, the pressure control data transmitted from the main control unit 12C to the pressure control unit 12D indicates that the pressure of the air inside of the inflatable bladder 2X is to be increased to about 13 mmHg within several seconds from when the first preliminary processing is started. The amount of the air inside of the inflatable bladder 2X in this embodiment is maintained as it is after the pressure of the air inside of the inflatable bladder 2X is once increased to about 13 mmHg. That is, after the pressure of the air inside of the inflatable bladder 2X reaches 13 mmHg, the pressure pump 11 stops the drive until the first preliminary processing is ended.

The processing of increasing the pressure of the air inside of the inflatable bladder 2X to 13 mmHg is performed as follows. Briefly speaking, the pressure control unit 12D causes the pressure pump 11 to perform any one of operations of injecting air into the inflatable bladder 2X, discharging the air, and stopping both processes of injecting and discharging the air, while monitoring, substantially in real time, the pressure of the air inside of the inflatable bladder 2X provided in the pressure applying belt 2. The pressure measuring unit 13 continuously generates the pressure data indicating the pressure of the air inside of the inflatable bladder 2X at that time point, and continuously transmits the generated pressure data to the pressure control unit 12D which has received the pressure data via the interface 104 and the input unit 12A. The pressure control unit 12D can grasp the pressure of the air inside of the inflatable bladder 2X at that time point based on the received pressure data. When the pressure falls below 13 mmHg, the pressure control unit 12D generates the first control data indicating that the air is required to be supplied into the inflatable bladder 2X, and transmits the first control data to the pressure pump 11 via the output unit 12B and the interface 104. In this manner, the pressure pump 11 supplies air into the inflatable bladder 2X, and hence the pressure of the air inside of the inflatable bladder 2X increases. As this processing continues, the pressure of the air inside of the inflatable bladder 2X gradually increases. When the pressure of the air inside of the inflatable bladder 2X indicated by the pressure data generated by the pressure measuring unit 13 indicates 13 mmHg, the pressure control unit 12D generates the first control data indicating that the pressure pump 11 is to be stopped, and transmits the first control data to the pressure pump 11. In this manner, the pressure pump 11 is stopped. By any chance, when the pressure of the air inside of the inflatable bladder 2X indicated by the pressure data generated by the pressure measuring unit 13 indicates a numerical value exceeding 13 mmHg, the pressure control unit 12D generates the first control data indicating that an operation of discharging air inside of the inflatable bladder 2X is to be performed by the pressure pump 11, and transmits the first control data to the pressure pump 11. In this manner, the pressure pump 11 discharges the air inside of the inflatable bladder 2X, and thus the pressure of the air inside of the inflatable bladder 2X decreases. As described above, the pressure control unit 12D causes the pressure of the air inside of the inflatable bladder 2X to reach 13 mmHg. The processing of discharging the air inside of the inflatable bladder 2X may be performed by the proportional valve 15 instead of the pressure pump 11. Such a method of controlling the pressure by the pressure control unit 12D is similarly performed in any of the cases of the second preliminary processing, the pre-processing, the occlusion processing, and the post-processing.

Under this state, the examiner adjusts the arm tightening degree obtained by the pressure applying belt 2 worn on a predetermined part of the subject, to thereby adjust the pressure applied to the subject's arm by the pressure applying belt 2 to become a predetermined certain wearing pressure of, for example, about 40 mmHg, including the above-mentioned air pressure of 13 mmHg. More precisely, the wearing pressure applied to the arm by the pressure applying belt 2 excluding the tightening force based on the pressure of the air inside of the inflatable bladder 2X is about 27 mmHg in this case. The wearing pressure is an initial pressure applied to the subject's arm by the pressure applying belt 2, and corresponds to a so-called zero of a pressure to be applied to the subject's arm by the pressure applying belt 2 due to the variation of the pressure of the air inside of the inflatable bladder 2X thereafter. The wearing pressure is not required to be about 40 mmHg, and may be larger or smaller. For example, when the subject is in a posture with no fear of dropping the pressure applying belt 2 from the subject's arm, for example, the subject is in a supine position and extends his or her arm on the floor, the wearing pressure may be 0 mmHg.

For example, the wearing pressure at the current time point can be checked as follows. As described above, in this embodiment, the pressure measuring unit 13 continuously transmits the pressure data to the pressure control unit 12D. In this manner, the pressure control unit 12D can always grasp the pressure of the air inside of the inflatable bladder 2X at that time point. In addition, in this embodiment, the pressure control unit 12D continuously transmits, to the main control unit 12C, the data indicating the pressure of the air inside of the inflatable bladder 2X at that time point, which is grasped by the pressure data. This processing may be achieved as processing of transferring, by the pressure control unit 12D, the pressure data received from the pressure measuring unit 13 to the main control unit 12C. In any case, the main control unit 12C can grasp the pressure of the air inside of the inflatable bladder 2X similarly to the pressure control unit 12D. The main control unit 12C which has received the data generates data identifying a numerical value indicating the pressure of the air inside of the inflatable bladder 2X at that time point, and transmits the data to the output unit 12B. The output unit 12B generates image data for displaying the numerical value on the display unit 17, and transmits the image data to the display unit 17 via the interface 104. The display unit 17 which has received the image data displays the image based on the image data.

As described above, when the first preliminary processing is executed, the display unit 17 continuously displays the pressure of the air inside of the inflatable bladder 2X substantially in real time. The examiner adjusts the tightening degree of the pressure applying belt 2 so that the pressure of the air inside of the inflatable bladder 2X at that time point, which is displayed on the display unit 17, becomes an appropriate value, in this embodiment, 40 mmHg, although not limited thereto. As described above, after the pressure of the air inside of the inflatable bladder 2X is increased to 13 mmHg, the drive of the pressure pump 11 is stopped under this state. Accordingly, when the pressure applying belt 2 is tightened, the pressure of the air inside of the inflatable bladder 2X squeezed between the tightening band 8 and the arm increases, and conversely the pressure of the air inside of the inflatable bladder 2X decreases when the pressure applying belt 2 is loosened. While viewing the numerical value displayed on the display unit 17, which changes depending on the tightening degree of the pressure applying belt 2, the examiner adjusts the tightening degree of the pressure applying belt 2.

After the adjustment of the tightening degree of the pressure applying belt 2 is ended, the examiner operates the operation unit 16 to end the first preliminary processing. Data indicating that the first preliminary processing is to be ended is transmitted to the main control unit 12C similarly to the data transmitted when the first preliminary processing is to be executed. The main control unit 12C transmits an instruction to end the first preliminary processing to the pressure control unit 12D. The pressure control unit 12D which has received the instruction generates the first control data indicating that the air inside of the inflatable bladder 2X is to be discharged until the pressure of the air inside of the inflatable bladder 2X becomes, for example, a normal pressure, and transmits the first control data to the pressure pump 11. The pressure pump 11 which has received the first control data discharges the air inside of the inflatable bladder 2X. In this manner, the first preliminary processing is ended.

Next, the second preliminary processing is executed. In each processing step at least after the second preliminary processing, the subject is kept in a resting state.

The second preliminary processing is processing for determining an initial value of the pressure of the air inside of the inflatable bladder 2X at the time when the pre-processing to be described later is started.

In order to execute the second preliminary processing, the examiner operates the operation unit 16 to perform input for causing the evaluation device 1 to execute the second preliminary processing. When this input is performed, data for executing the second preliminary processing is transmitted to the main control unit 12C similarly to the data transmitted when the first preliminary processing is to be executed. The main control unit 12C which has received the data transmits information indicating that the second preliminary processing is to be executed to the pressure control unit 12D and the pulse wave measuring unit 12F. The main control unit 12C further generates pressure application control data in the case of executing the second preliminary processing, and transmits the pressure application control data to the pressure control unit 12D. The pressure application control data at this time is data as described later.

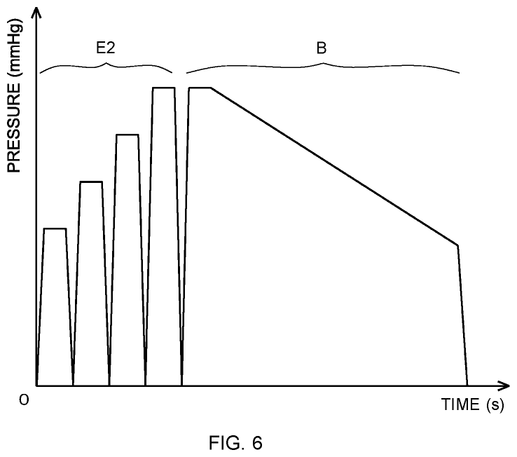

In the time chart of FIG. 6, a part denoted by the reference symbol E2 represents a period of time in which the second preliminary processing is executed. In FIG. 6, the horizontal axis represents an elapse of time from a time point at which the second preliminary processing is started, and the vertical axis represents the pressure of the air inside of the inflatable bladder 2X of the pressure applying belt 2.

In the second preliminary processing in this embodiment, the pressure of the air inside of the inflatable bladder 2X of the pressure applying belt 2 is gradually increased in stages as illustrated in FIG. 6. Changing the pressure of the air of the inflatable bladder 2X over time as described above (gradually increasing the pressure in stages) is the content of the pressure application control data in the second preliminary processing, which is generated by the main control unit 12C and transmitted to the pressure control unit 12D. However, the pressure application control data at the time of the second preliminary processing in this embodiment does not identify to which stage the pressure of the air inside of the inflatable bladder 2X is to be increased. This point is described later.

The pressure of the air inside of the inflatable bladder 2X is, for example, 0 mmHg at the first time point of the second preliminary processing, and is increased to a predetermined pressure in several seconds so that pressure application is performed in a first stage. The pressure of the air inside of the inflatable bladder 2X at this time can be a pressure that is lower by some extent, for example, by about 30 mmHg from a pressure which is thought to cause no tension to the vascular wall of the subject's artery. As described above, no tension is applied to the vascular wall of the subject's artery when a pressure around a subject's mean blood pressure is applied to the subject's arm. Accordingly, for example, the blood pressure of the subject can be measured in advance, and a pressure lower to some extent from the subject's mean blood pressure can be determined as the pressure in the first stage described above. When the input of the data of the subject's mean blood pressure is allowed to be performed from the operation unit 16, the main control unit 12C which has received the data can determine the pressure in the first stage depending on the subject's mean blood pressure, and generate the pressure application control data at the time of the second preliminary processing in consideration thereof. The pressure in the pressure application in the first stage may be determined based on the knowledge, the experience, or the like of the examiner regardless of the subject's mean blood pressure.

The pressure application in the first stage is continued at the same pressure for a predetermined time period, for example, 10 seconds. After that, the pressure application in the first stage is ended, and the pressure of the air inside of the inflatable bladder 2X is dropped to 0 mmHg in several seconds. Then, pressure application in a second stage is performed. The pressure application in the second stage is performed immediately after the pressure application in the first stage is performed, and the pressure of the air inside of the inflatable bladder 2X, which has been 0 mmHg, is increased to a predetermined pressure in several seconds. The pressure of the air inside of the inflatable bladder 2X in the second stage is set to be higher than the pressure of the air inside of the inflatable bladder 2X in the first stage by some extent, for example, by about 10 mmHg. The pressure application in the second stage is continued at the same pressure for a predetermined time period, in general, 10 seconds, which is the same as the case of the pressure application in the first stage. After that, the pressure application in the second stage is ended. Similarly, pressure application in a third stage, pressure application in a fourth stage, are repeatedly executed a required number of times. Description of the “required number of times” is given later.

As described above, an internal pressure caused by the blood pressure is applied to the vascular wall of the subject's artery. In contrast, when a pressure is applied to the arm from the outer side, this pressure acts as an external pressure with respect to the vascular wall of the artery. When the internal pressure and the external pressure are balanced, there is achieved a state in which no tension is applied to the vascular wall. Pulse waves are caused in the artery by the heartbeat. The pulse wave amplitude is maximum when no tension is applied to the vascular wall, and the pulse wave amplitude is decreased whether the internal pressure is larger or smaller than that time. Roughly speaking, when the pressure applied to the arm by the pressure applying belt 2 is plotted in the horizontal axis, and the pulse wave amplitude is plotted in the vertical axis, the pulse wave amplitude draws a parabola which opens downward and has a vertex when the pressure applied to the arm by the pressure applying belt 2 becomes a magnitude balanced with the internal pressure applied to the vascular wall.

As described above, when the pressure of the air inside of the inflatable bladder 2X is increased in stages, the pressure applied to the arm by the pressure applying belt 2 eventually exceeds the internal pressure applied to the vascular wall. Finding out this pressure is an object of the second preliminary processing.

While the second preliminary processing is executed, as described above, the pressure data is always repeatedly input to the pulse wave measuring unit 12F. Accordingly, the pulse wave measuring unit 12F is in a state of always monitoring the pulse wave amplitude at that time point. The pressure data includes, in addition to data of a large air pressure change caused by the injection or discharge of air to or from the inflatable bladder 2X by the pressure pump 11, data of a very minute air pressure variation caused by the pulse wave amplitude of the pulse wave of the subject. The pulse wave measuring unit 12F which has received the pressure data detects the pulse wave amplitude from the data of the minute air pressure variation included in the pressure data. When the pressure application to the arm in the first stage is continued at the same pressure, basically, or ideally, the pulse wave amplitude remains the same. The same holds true also when the pressure application in the second stage, the pressure application in the third stage, are executed. The pulse wave measuring unit 12F detects the pulse wave amplitude at the time when the pressure application in the first stage is performed, and also detects the pulse wave amplitude at the time when the pressure application in the second stage, the pressure application in the third stage, are performed. As described above, the pulse wave amplitude is increased as the tightening pressure of the pressure applying belt 2 with respect to the arm, which is gradually increased every time the stage is raised, comes closer to the internal pressure applied to the vascular wall, and the pulse wave amplitude is decreased when the tightening pressure exceeds the internal pressure applied to the vascular wall. When, in a certain stage, the pulse wave amplitude becomes smaller than that in the previous stage, the pulse wave measuring unit 12F generates data identifying the pressure of the air inside of the inflatable bladder 2X at this time point, and transmits the generated data to the main control unit 12C. When the pressure identified by this data is set as the pressure of the air inside of the inflatable bladder 2X, the pressure applied to the arm by the pressure applying belt 2 always exceeds the pressure at which the pulse wave amplitude in the subject's artery is maximum (no tension is applied to the vascular wall of the artery).

When the main control unit 12C receives this data, the main control unit 12C generates data indicating that the second preliminary processing is to be ended, and transmits this data to the pressure control unit 12D. When receiving this data, the pressure control unit 12D ends the second preliminary processing. That is, the pressure application in the next stage is not performed. That is, the “required number of times” of pressure application is ended.

In this embodiment, after the second preliminary processing is ended, the main control unit 12C automatically and successively starts the pre-processing. It is not always required to automatically and successively perform the second preliminary processing and the pre-processing. An interval may be given between both of the processing steps, or the pre-processing may be started on condition that the operation unit 16 is operated by the examiner. As the interval between both the processing steps is shorter, the time required for evaluating the vascular endothelial function is reduced, and thus the subject's burden is reduced. The same circumstance holds true for intervals between other processing steps. The pre-processing is processing for identifying the pulse wave amplitude under a state in which no tension is applied to the vascular wall in the resting state. This processing has the same purpose as the processing of identifying the pulse wave amplitude in the resting state in the ezFMD.

When the pre-processing is to be executed, the main control unit 12C transmits information indicating that the pre-processing is to be executed to the pressure control unit 12D, the maximum pulse wave pressure identifying unit 12E, and the pulse wave measuring unit 12F. The main control unit 12C further generates pressure application control data in the case of executing the pre-processing, and transmits the pressure application control data to the pressure control unit 12D. The pressure application control data at this time is data as described later.

In the time chart of FIG. 6, a part denoted by the reference symbol B represents a period of time in which the pre-processing is executed.

In the pre-processing in this embodiment, as illustrated in FIG. 6, for example, the pressure of the air inside of the inflatable bladder 2X of the pressure applying belt 2 is steeply increased within about 3 seconds to about 5 seconds, and is then smoothly and gradually decreased. A period of time in which the pressure of the air inside of the inflatable bladder 2X is smoothly decreased is a state in which a first phase referred to in the subject application is executed. Changing the pressure of the air of the inflatable bladder 2X over time as described above (once steeply increasing the pressure and then gradually decreasing the pressure) is the content of the pressure application control data in the pre-processing, which is generated by the main control unit 12C and transmitted to the pressure control unit 12D. The pressure of the air inside of the inflatable bladder 2X at the first time point of the pre-processing is equal to the pressure obtained at the final stage at the time of the second preliminary processing. As described above, in the second preliminary processing, the data identifying the pressure of the air inside of the inflatable bladder 2X in the stage in which the pulse wave amplitude is decreased as compared to that in the previous stage is transmitted from the pulse wave measuring unit 12F to the main control unit 12C. The main control unit 12C generates the pressure application control data so that the pressure identified by this data becomes the pressure at the first time point of the pre-processing.

In the pre-processing, the pressure of the air inside of the inflatable bladder 2X is gradually decreased from the pressure at the first time point. The pressure inside of the inflatable bladder 2X is decreased until the tightening force applied to the arm by the pressure applying belt 2 falls below the pressure at which the pulse wave amplitude in the subject's artery is maximum (no tension is applied to the vascular wall of the artery). That is, the change of the pressure performed in the pre-processing passes the pressure at which the pulse wave amplitude is maximum. As described above, the pressure of the air inside of the inflatable bladder 2X at the first time point of the pre-processing is set so that the pressure applied to the arm by the pressure applying belt 2 definitely exceeds the pressure at which the pulse wave amplitude in the subject's artery is maximum (no tension is applied to the vascular wall of the artery). Accordingly, when the pressure of the air inside of the inflatable bladder 2X is decreased so as to certainly fall below the pressure at which the pulse wave amplitude is maximum (no tension is applied to the vascular wall of the artery), the pressure applied to the arm by the pressure applying belt 2, which changes in the pre-processing, always passes the pressure at which the pulse wave amplitude is maximum. The lowest pressure of the gas inside of the inflatable bladder 2X in the pre-processing can be set to, for example, the normal pressure, or can be set to the pressure in the first stage in the second preliminary processing. In this embodiment, the latter pressure is basically adopted, but in order to give some margin, the pressure inside of the inflatable bladder 2X is decreased to a pressure slightly lower than the latter pressure. In order to similarly give some margin, the pressure inside of the inflatable bladder 2X at the first time point of the pre-processing, more precisely, at the first time point of the first phase may be set to be slightly higher by, for example, about 10 mmHg than the pressure applied to the air inside of the inflatable bladder 2X in the final stage of the second preliminary processing.

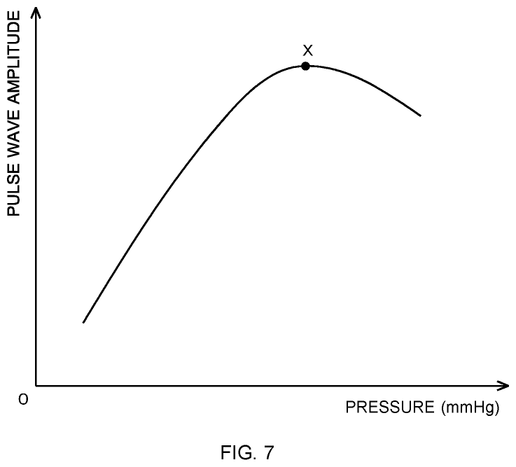

While the pre-processing is executed, in more detail, at least while the pressure of the air inside of the inflatable bladder 2X is smoothly decreased, the pressure data is always repeatedly input to the pulse wave measuring unit 12F. Accordingly, the pulse wave measuring unit 12F is in a state of always monitoring the pulse wave amplitude at that time point. As described above, pulse waves are caused in the artery by the heartbeat. The pulse wave amplitude is maximum when no tension is applied to the vascular wall, and the pulse wave amplitude is decreased whether the internal pressure is larger or smaller than that time. As the pressure inside of the inflatable bladder 2X is decreased, as shown in FIG. 7, the pulse wave amplitude draws a convex upward parabola graph in which the pulse wave amplitude is increased until the pressure of the air inside of the inflatable bladder 2X reaches a certain value, and is thereafter decreased. In the pre-processing, this graph is drawn from the right side.

The pulse wave measuring unit 12F generates the pulse wave amplitude data being data identifying the pulse wave amplitude at the time point at which the pulse wave amplitude is maximum (at the time point of X of FIG. 7). The pulse wave amplitude data is transmitted from the pulse wave measuring unit 12F to the output unit 12B. The output unit 12B transmits the pulse wave amplitude data generated at the time of the pre-processing to the recording unit 18, and records the pulse wave amplitude data in the recording unit 18.

Meanwhile, the pulse wave measuring unit 12F transmits the data identifying the timing at which the pulse wave amplitude is maximum to the maximum pulse wave pressure identifying unit 12E. The maximum pulse wave pressure identifying unit 12E which has received the data generates the maximum pulse wave pressure data being data identifying the maximum pulse wave pressure corresponding to the pressure inside of the inflatable bladder 2X at the time point at which the pulse wave amplitude is maximum in the pre-processing. The generated maximum pulse wave pressure data is transmitted from the maximum pulse wave pressure identifying unit 12E to the main control unit 12C. The main control unit 12C keeps this data until the post-processing is started.

As described above, the pre-processing is ended.

In this embodiment, when the pre-processing is executed, the pressure of the air inside of the inflatable bladder 2X is changed in a direction of decreasing the pressure, but the pressure of the air inside of the inflatable bladder 2X may be changed in a direction of increasing the pressure. However, in an actual case, when the processing of injecting air into the inflatable bladder 2X and the processing of discharging air from the inflatable bladder 2X are compared with each other, the latter processing can more easily attain a smooth pressure change. Accordingly, when the latter processing is adopted, it becomes easier to make the pulse wave amplitude data identified at the time of the pre-processing more accurate.

Next, the occlusion processing is executed.

In this embodiment, after the pre-processing is ended, the main control unit 12C automatically and successively starts the occlusion processing. It is not required to automatically and successively perform the pre-processing and the occlusion processing as it is not required to automatically and successively perform the second preliminary processing and the pre-processing. The occlusion processing is processing of causing occlusion (state in which the arterial blood is completely blocked) in the artery of the arm to which the pressure applying belt 2 is attached, and also releasing the occlusion. This processing has the same purpose as the processing of identifying the pulse wave amplitude in the resting state in the ezFMD.

When the occlusion processing is to be executed, the main control unit 12C transmits information indicating that the occlusion processing is to be executed to the pressure control unit 12D. The main control unit 12C further generates pressure application control data in the case of executing the occlusion processing, and transmits the pressure application control data to the pressure control unit 12D. The pressure application control data at this time is data as described later.

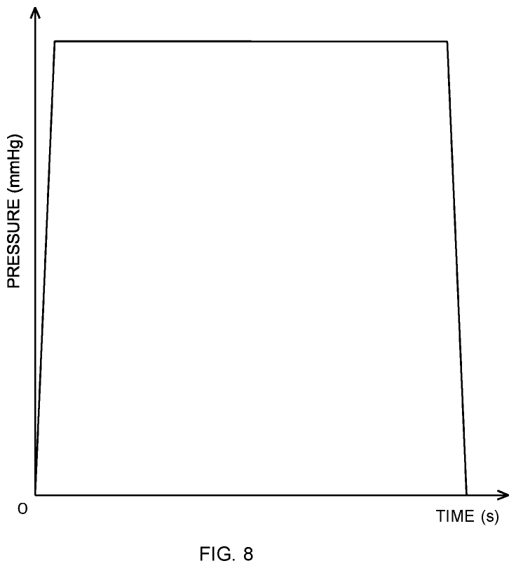

FIG. 8 shows a time chart of a period of time of the occlusion processing. The horizontal axis and the vertical axis are the same as those of FIG. 6.

In the occlusion processing in this embodiment, as illustrated in FIG. 8, for example, the pressure of the air inside of the inflatable bladder 2X of the pressure applying belt 2 is steeply increased within about 3 seconds to about 5 seconds, and then the pressure is kept for at least 3 minutes. After that, the pressure is decreased to the normal pressure in about several seconds.

The pressure of the air inside of the inflatable bladder 2X when the pressure is kept constant is equal to or larger than a pressure at which the pressure applying belt 2 causes occlusion in the artery of the arm to which the pressure applying belt 2 is attached. A period of time in which the pressure of the air inside of the inflatable bladder 2X is kept constant and is then decreased to the normal pressure is a period of time in which a second phase in the subject application is executed.

In theory, when the pressure applying belt 2 applies a tightening force at the same pressure as the subject's systolic blood pressure to the arm, the occlusion occurs in the artery of the arm. However, in order to give some margin to reliably cause the occlusion, this pressure may be required to be larger than the same pressure as the subject's systolic blood pressure. For example, the subject's systolic blood pressure may be input to the evaluation device 1 by the examiner operating the operation unit 16. In this manner, the main control unit 12C which has received the subject's systolic blood pressure can generate the pressure application control data reflecting the level of the systolic blood pressure. When it is intended that the evaluation result of the vascular endothelial function obtained by the general FMD or the ezFMD and the evaluation result of the evaluation system of this embodiment be allowed to be easily compared with each other, this pressure is required to be a pressure higher by 50 mmHg than the subject's systolic blood pressure. In general, the systolic blood pressure is obtained by adding, to the mean blood pressure, a pressure of about ⅔ of a difference between the systolic blood pressure and the diastolic blood pressure. In view of the above, the pressure of the air inside of the inflatable bladder 2X in the second phase may also be determined based on the mean blood pressure as, for example, “mean blood pressure +80 mmHg.” When it is guaranteed that the occlusion is continuously caused, the pressure of the air inside of the inflatable bladder 2X at the time of the occlusion or when the second phase is executed may fluctuate.

The occlusion time period is set to 3 minutes or more because this time period is sufficient for causing a change of the elasticity of the blood vessel before and after the occlusion based on release of nitric oxide. This time period may be larger, for example, 5 minutes similarly to the case of the general FMD or the ezFMD. In this manner, the evaluation result of the vascular endothelial function obtained by the evaluation device 1 can be more easily compared with the evaluation result obtained by the general FMD or the ezFMD.

To which value the air pressure inside of the inflatable bladder 2X is decreased when the occlusion is released may be determined as appropriate within a range in which the occlusion can be released. In this embodiment, the pressure of the air inside of the inflatable bladder 2X is decreased to the normal pressure, but an air pressure of about 20 mmHg may remain inside the inflatable bladder 2X even at the stage at which the occlusion is released, although it is quite meaningless.

In the occlusion processing, the measurement of the pulse wave amplitude is not required to be performed.

In this embodiment, the occlusion processing is ended after the pressure of the air inside of the inflatable bladder 2X is recovered to the normal pressure.

When the occlusion processing is executed, the subject may perform light training under a state in which the occlusion is caused in the artery of the arm. In this manner, there is a possibility that nitric oxide in the blood may be further increased so that the change of the elasticity of the blood vessel between the time point at which the pre-processing is executed and the time point at which the post-processing is executed can be further increased. In this case, the pressure applying belt 2 is kept in a state of being connected to the evaluation device 1. When the subject performs exercise under this state, in some cases, the arm muscle may be thickened, and the pressure of the air inside of the inflatable bladder 2X may be increased. In such a case, the pressure control unit 12D can generate the second control data to drive the proportional valve 15, thereby being capable of keeping the pressure inside of the inflatable bladder 2X constant. Specifically, the pressure control unit 12D which has generated the second control data in accordance with the instruction from the main control unit 12C transmits the second control data to the proportional valve 15 via the output unit 12B. The proportional valve 15 receives the second control data, and executes the above-mentioned processing based on the second control data. When such processing is not required, the proportional valve 15 is not required. However, when the subject performs exercise during the occlusion processing, the evaluation result of the vascular endothelial function obtained by the evaluation device 1 is difficult to be compared with the evaluation result obtained by the general FMD or the ezFMD, and is required be thought as a different thing.

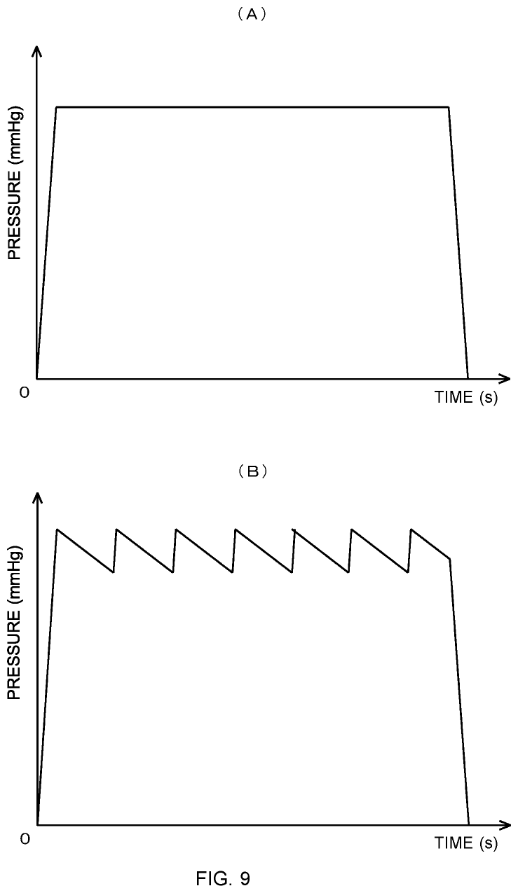

In this embodiment, after the occlusion processing is ended, the main control unit 12C automatically starts the post-processing. It is preferred that a time interval from the end of the occlusion processing to the start of the post-processing, more precisely, an interval from when the occlusion processing is ended to when the graph of FIG. 9 starts to rise be at least about 10 seconds, for example, from about 15 seconds to about 30 seconds. When the tightening force is applied to the arm by the pressure applying belt 2 immediately after the occlusion processing is ended, the occurrence of the shear stress in the artery based on an increase of a blood flow due to the release of the occlusion, and the production of nitric oxide based on the occurrence of the shear stress may become insufficient. As a result, there is a fear in that the improvement of the elasticity of the artery based on the production of the nitric oxide becomes insufficient. It is not required to automatically and successively perform the occlusion processing and the post-processing similarly to the above. The post-processing is processing for identifying the pulse wave amplitude under a state in which no tension is applied to the vascular wall after the release of the occlusion. This processing has the same purpose as the processing of identifying the pulse wave amplitude after the release of the occlusion in the ezFMD.

When the post-processing is to be executed, the main control unit 12C transmits information indicating that the post-processing is to be executed to the pressure control unit 12D and the pulse wave measuring unit 12F. The main control unit 12C further generates pressure application control data in the case of executing the post-processing, and transmits the pressure application control data to the pressure control unit 12D. The pressure application control data at this time is data as described later.

FIG. 9 shows a time chart of a period of time of the post-processing. The horizontal axis and the vertical axis are the same as those of FIG. 6.

In the post-processing in this embodiment, as illustrated in FIG. 9(A), for example, the pressure of the air inside of the inflatable bladder 2X of the pressure applying belt 2 is steeply increased within about 3 seconds to about 5 seconds, and then the pressure is kept constant until at least 90 seconds elapse from when the occlusion is released (in this embodiment, as this time period, the pressure is kept constant until 120 seconds elapse). After that, the pressure is decreased to the normal pressure in about several seconds.

A period of time in which the pressure inside of the inflatable bladder 2X is kept constant is a period of time in which a third phase referred to in the subject application is executed. The pressure of the air inside of the inflatable bladder 2X at the time when the pressure is kept constant is, in this embodiment, the maximum pulse wave pressure identified in the pre-processing. As described above, the main control unit 12C keeps the maximum pulse wave pressure data until the post-processing is started, and hence the main control unit 12C can generate the pressure application control data reflecting the maximum pulse wave pressure. In theory, when the pressure of the air inside of the inflatable bladder 2X is kept to the maximum pulse wave pressure, the tightening force applied to the subject's arm by the pressure applying belt 2 causes a state in which no tension is applied to the vascular wall of the arm artery. This state causes a larger pulse wave amplitude than that of a state in which tension is applied to the vascular wall of the artery. Thus, it is considered that the change of the pulse wave amplitude based on the improvement of the elasticity of the blood vessel based on the rise of the nitric oxide concentration in the blood after the release of the occlusion can be more effectively detected. The time period from when the occlusion is released to when the artery attains the highest elasticity is, as described above, within an elapse of 45 seconds to an elapse of 120 seconds from the release of the occlusion, and there are individual differences. However, starting the time in which no tension is applied to the vascular wall of the artery within 45 seconds from the release of the occlusion in the post-processing can be easily achieved when the time period from the end of the occlusion processing to the moment of rise of the graph of FIG. 9(A) is set to from about 30 seconds to about 40 seconds at maximum, and when the time required to steeply increase the pressure of the air inside of the inflatable bladder 2X immediately after the post-processing is started is reduced. For example, in this embodiment, in order to give some margin, the pressure of the air inside of the inflatable bladder 2X reaches the maximum pulse wave pressure within 35 seconds to seconds from the release of the occlusion. Further, as described above, in this embodiment, the pressure of the air inside of the inflatable bladder 2X is kept to the maximum pulse wave pressure for a time period until 120 seconds elapse from the release of the occlusion. As described above, in this embodiment, the pulse wave amplitude measured in the period of time in which the pressure of the air inside of the inflatable bladder 2X is kept to the maximum pulse wave pressure is a pulse wave amplitude of a pulse wave caused in the artery in which no tension is applied to the vascular wall.

In this embodiment, the pressure of the air inside of the inflatable bladder 2X, which is steeply increased after the post-processing is started and is then kept constant, is set to be equal to the maximum pulse wave pressure as described above. However, this pressure may be constant within a range of plus and minus 15 mmHg around the maximum pulse wave pressure, or, for example, as illustrated in FIG. 9(B), this pressure may fluctuate within a range of plus and minus 15 mmHg around the maximum pulse wave pressure. When the pressure of the air inside of the inflatable bladder 2X is kept at such a pressure, in theory, the tension applied to the vascular wall of the artery is relatively small, and hence the pulse wave amplitude measured in the post-processing is sufficiently large.

While the post-processing is executed, in more detail, while the pressure of the air inside of the inflatable bladder 2X is kept to achieve a state in which no tension or a relatively small tension is applied to the vascular wall of the arm artery, the pressure data is always repeatedly input to the pulse wave measuring unit 12F. Accordingly, the pulse wave measuring unit 12F is in a state of always monitoring the pulse wave amplitude at that time point. The elasticity of the vascular wall changes from moment to moment due to the influence of the nitric oxide produced in the blood due to the influence of the release of the occlusion. The pulse wave amplitude also changes based on this change.

The pulse wave measuring unit 12F generates the pulse wave amplitude data being data identifying the pulse wave amplitude at the time point at which the pulse wave amplitude is maximum. The pulse wave amplitude data is transmitted from the pulse wave measuring unit 12F to the output unit 12B. The output unit 12B transmits the pulse wave amplitude data generated at the time of the post-processing to the recording unit 18, and records the pulse wave amplitude data in the recording unit 18. The two pieces of pulse wave amplitude data obtained in the series of pre-processing and post-processing are recorded in the recording unit 18 as a pair. The two pieces of pulse wave amplitude data obtained in the series of pre-processing and post-processing may be collectively transmitted to the recording unit 18 to be recorded in the recording unit 18.

As described above, the post-processing is ended.

In this manner, for now, the evaluation of the vascular endothelial function by the vascular endothelial function evaluation system is ended.

The examiner can use the pulse wave amplitudes identified by the pair of pieces of pulse wave amplitude data recorded in the recording unit 18 to evaluate the vascular endothelial function of a subject being a target of recording those pulse wave amplitudes. When the pulse wave amplitude recorded in the pre-processing is represented by P_{B}, and the pulse wave amplitude recorded in the post-processing is represented by P_{A}, the examiner may calculate P_{A}/P_{B} or (P_{A}−P_{B})/P_{B}, thereby being capable of obtaining the evaluation result of the vascular endothelial function.

Meanwhile, the evaluation of the vascular endothelial function can also be performed by the evaluation device 1, which is allowed in this embodiment. When the evaluation device 1 is caused to create the evaluation result of the vascular endothelial function, the examiner operates the operation unit 16 to identify a pair of pieces of pulse wave amplitude data for the subject being the evaluation target. In this case, the pair of pieces of pulse wave amplitude data is read out from the recording unit 18, and is received by the main control unit 12C via the interface 104 and the input unit 12A. The main control unit 12C performs calculation as described above based on those two pieces of pulse wave amplitude data, and generates the evaluation data being data of the evaluation result of the vascular endothelial function. The main control unit 12C transmits the generated evaluation data to the output unit 12B. After receiving the evaluation data, the output unit 12B generates the image data of an image including, for example, characters, for use to display the evaluation data on the display unit 17, and transmits the image data to the display unit 17. The display unit 17 which has received the image data displays the image based on the image data. When the image is viewed, the evaluation result of the subject's vascular endothelial function is understood.

In this embodiment, the generation of the pair of pieces of pulse wave amplitude data and the recording to the recording unit 18, and the generation of the evaluation data are performed separately on the condition that data for prompting generation of the evaluation data is input from the operation unit 16, but the present invention is not always limited thereto. For example, when the pre-processing and the post-processing are executed or after those processing steps are executed, the main control unit 12C may receive the pieces of pulse wave amplitude data from the pulse wave measuring unit 12F, and the main control unit 12C which has received those pieces of pulse wave amplitude data may generate the evaluation data based on the two pieces of pulse wave amplitude data received from the pulse wave measuring unit 12F, without reading out the pieces of pulse wave amplitude data from the recording unit 18. In this case, the pieces of pulse wave amplitude data being paired are not required to be recorded in the recording unit 18. How to treat the generated evaluation data may be the same as that in the case described above.

### REFERENCE SIGNS LIST

1 vascular endothelial function evaluation device
2 pressure applying belt
3 rubber tube
8 tightening band
9 valved coupler
11 pressure pump
12 control unit
12A input unit
12B output unit
12C main control unit
12D pressure control unit
12E maximum pulse wave pressure identifying unit
12F pulse wave measuring unit
15 proportional valve
16 operation unit
17 display unit
18 recording unit
22 battery

1. A vascular endothelial function evaluation device, which is configured to form a vascular endothelial function evaluation system in combination with:
a tightener including:
a belt having a length which allows the belt to be wrapped around a predetermined part of any one of limbs;
fixing means for fixing the belt under a state in which the belt is wrapped around the predetermined part of the limb; and
an inflatable bladder which is provided on the belt, and is configured to apply a predetermined tightening pressure to the predetermined part of the limb by tightening the predetermined part of the limb through loading of a gas inside of the inflatable bladder under a state in which the belt wrapped around the predetermined part of the limb is fixed by the fixing means;
a pressure varying device configured to set a pressure of the gas inside of the inflatable bladder to a desired pressure; and
a pulse wave measuring device configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter,
the vascular endothelial function evaluation device comprising control means for controlling the pressure varying device, the control means being configured to receive the pulse wave data from the pulse wave measuring device,
wherein the control means is configured to execute:
pre-processing of controlling the pressure varying device so as to cause the pressure varying device to execute a first phase being processing of changing the pressure of the gas inside of the inflatable bladder to pass a range in which the pulse wave amplitude is assumed to be maximum, receiving the pulse wave data a plurality of times from the pulse wave measuring device while the pressure inside of the inflatable bladder is changed by the first phase to identify a maximum pulse wave pressure being the pressure of the gas inside of the inflatable bladder at a time when the pulse wave amplitude is maximum, and recording the pulse wave amplitude at the time when the maximum pulse wave pressure is caused;
occlusion processing of controlling the pressure varying device so as to cause the pressure varying device to execute a second phase being processing of maintaining the pressure of the gas inside of the inflatable bladder for at least 3 minutes or more at a pressure equal to or higher than a pressure at which occlusion occurs in the artery of the limb to which the tightener is fixed, and then decreasing the pressure of the gas inside of the inflatable bladder; and
post-processing of controlling the pressure varying device so as to cause the pressure varying device to execute a third phase of maintaining the pressure of the gas inside of the inflatable bladder within a range of 15 mmHg around the maximum pulse wave pressure until at least 90 seconds elapse from when the processing of decreasing the pressure of the gas inside of the inflatable bladder is ended in the occlusion processing, receiving the pulse wave data a plurality of times from the pulse wave measuring device while the pressure inside of the inflatable bladder is maintained in a state of the third phase, and recording a maximum pulse wave amplitude based on pieces of pulse wave data received the plurality of times.

2. The vascular endothelial function evaluation device according to claim 1, wherein, in the third phase, the pressure of the gas inside of the inflatable bladder is kept constant within the range of 15 mmHg around the maximum pulse wave pressure.

3. The vascular endothelial function evaluation device according to claim 1, wherein, in the third phase, the pressure of the gas inside of the inflatable bladder is kept constant at the maximum pulse wave pressure.

4. The vascular endothelial function evaluation device according to claim 1, wherein, in the third phase, the pressure of the gas inside of the inflatable bladder fluctuates within the range of 15 mmHg around the maximum pulse wave pressure.

5. The vascular endothelial function evaluation device according to claim 1, wherein the control means is configured to generate result data of a numerical value of a result of performing predetermined calculation based on the pulse wave amplitude recorded in the pre-processing and the pulse wave amplitude recorded in the post-processing.

6. The vascular endothelial function evaluation device according to claim 1, wherein the pulse wave measuring device is adapted to measure the pressure of the gas inside of the inflatable bladder as the predetermined parameter.

7. A vascular endothelial function evaluation system, comprising:
a tightener including:
a belt having a length which allows the belt to be wrapped around a predetermined part of any one of limbs;
fixing means for fixing the belt under a state in which the belt is wrapped around the predetermined part of the limb; and
an inflatable bladder which is provided on the belt, and is configured to apply a predetermined tightening pressure to the predetermined part of the limb by tightening the predetermined part of the limb through loading of a gas inside of the inflatable bladder under a state in which the belt wrapped around the predetermined part of the limb is fixed by the fixing means;
a pressure varying device configured to set a pressure of the gas inside of the inflatable bladder to a desired pressure;
a pulse wave measuring device configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter; and
control means for controlling the pressure varying device, the control means being configured to receive the pulse wave data from the pulse wave measuring device,
wherein the control means is configured to execute:
pre-processing of controlling the pressure varying device so as to cause the pressure varying device to execute a first phase being processing of changing the pressure of the gas inside of the inflatable bladder to pass a range in which the pulse wave amplitude is assumed to be maximum, receiving the pulse wave data a plurality of times from the pulse wave measuring device while the pressure inside of the inflatable bladder is changed by the first phase to identify a maximum pulse wave pressure being the pressure of the gas inside of the inflatable bladder at a time when the pulse wave amplitude is maximum, and recording the pulse wave amplitude at the time when the maximum pulse wave pressure is caused;
occlusion processing of controlling the pressure varying device so as to cause the pressure varying device to execute a second phase being processing of maintaining the pressure of the gas inside of the inflatable bladder for at least 3 minutes or more at a pressure equal to or higher than a pressure at which occlusion occurs in the artery of the limb to which the tightener is fixed, and then decreasing the pressure of the gas inside of the inflatable bladder; and
post-processing of controlling the pressure varying device so as to cause the pressure varying device to execute a third phase of maintaining the pressure of the gas inside of the inflatable bladder within a range of 15 mmHg around the maximum pulse wave pressure until at least 90 seconds elapse from when the processing of decreasing the pressure of the gas inside of the inflatable bladder is ended in the occlusion processing, receiving the pulse wave data a plurality of times from the pulse wave measuring device while the pressure inside of the inflatable bladder is maintained in a state of the third phase, and recording a maximum pulse wave amplitude based on pieces of pulse wave data received the plurality of times.

8. A vascular endothelial function evaluation method of a vascular endothelial function evaluation device,
the vascular endothelial function evaluation device being configured to form a vascular endothelial function evaluation system in combination with:
a tightener including:
a belt having a length which allows the belt to be wrapped around a predetermined part of any one of limbs;
fixing means for fixing the belt under a state in which the belt is wrapped around the predetermined part of the limb; and
an inflatable bladder which is provided on the belt, and is configured to apply a predetermined tightening pressure to the predetermined part of the limb by tightening the predetermined part of the limb through loading of a gas inside of the inflatable bladder under a state in which the belt wrapped around the predetermined part of the limb is fixed by the fixing means;
a pressure varying device configured to set a pressure of the gas inside of the inflatable bladder to a desired pressure; and
a pulse wave measuring device configured to measure, in a vicinity of a part of the limb at which the tightener is fixed or on a further distal end side of the limb from the part, a predetermined parameter varying in accordance with a variation of a magnitude of a pulse wave of an artery, and to generate pulse wave data of a pulse wave amplitude based on the predetermined parameter,
the vascular endothelial function evaluation device comprising control means for controlling the pressure varying device, the control means being configured to receive the pulse wave data from the pulse wave measuring device,
wherein the control means is configured to execute the vascular endothelial function evaluation method comprising:
pre-processing of controlling the pressure varying device so as to cause the pressure varying device to execute a first phase being processing of changing the pressure of the gas inside of the inflatable bladder to pass a range in which the pulse wave amplitude is assumed to be maximum, receiving the pulse wave data a plurality of times from the pulse wave measuring device while the pressure inside of the inflatable bladder is changed by the first phase to identify a maximum pulse wave pressure being the pressure of the gas inside of the inflatable bladder at a time when the pulse wave amplitude is maximum, and recording the pulse wave amplitude at the time when the maximum pulse wave pressure is caused;
occlusion processing of controlling the pressure varying device so as to cause the pressure varying device to execute a second phase being processing of maintaining the pressure of the gas inside of the inflatable bladder for at least 3 minutes or more at a pressure equal to or higher than a pressure at which occlusion occurs in the artery of the limb to which the tightener is fixed, and then decreasing the pressure of the gas inside of the inflatable bladder; and
post-processing of controlling the pressure varying device so as to cause the pressure varying device to execute a third phase of maintaining the pressure of the gas inside of the inflatable bladder within a range of 15 mmHg around the maximum pulse wave pressure until at least 90 seconds elapse from when the processing of decreasing the pressure of the gas inside of the inflatable bladder is ended in the occlusion processing, receiving the pulse wave data a plurality of times from the pulse wave measuring device while the pressure inside of the inflatable bladder is maintained in a state of the third phase, and recording a maximum pulse wave amplitude based on pieces of pulse wave data received the plurality of times.

9. The vascular endothelial function evaluation device according to claim 2, wherein the pulse wave measuring device is adapted to measure the pressure of the gas inside of the inflatable bladder as the predetermined parameter.

10. The vascular endothelial function evaluation device according to claim 3, wherein the pulse wave measuring device is adapted to measure the pressure of the gas inside of the inflatable bladder as the predetermined parameter.

11. The vascular endothelial function evaluation device according to claim 4, wherein the pulse wave measuring device is adapted to measure the pressure of the gas inside of the inflatable bladder as the predetermined parameter.

12. The vascular endothelial function evaluation device according to claim 5, wherein the pulse wave measuring device is adapted to measure the pressure of the gas inside of the inflatable bladder as the predetermined parameter.
